(19) United States
(12) Patent Application Publication
(10) Pub. No.: US 2017/0248575 A1
(43) Pub. Date: Aug. 31, 2017

(54) Method For Improved Accuracy Of Blood Testing

(71) Applicant: ESCA Tech, Inc., Milwaukee, WI (US)

(72) Inventor: Daniel P. Askin, Milwaukee, WI (US)

(21) Appl. No.: 15/584,707

(22) Filed: May 2, 2017

Related U.S. Application Data
(63) Continuation of application No. 11/487,183, filed on Jul. 13, 2006.
(60) Provisional application No. 60/699,286, filed on Jul. 14, 2005.

(51) Int. Cl. G01N 33/49; A61K 8/41; A61B 5/15; A61K 8/55; A61B 5/145; A61Q 19/10; A61K 8/92

(52) U.S. Cl. CPC G01N 33/49; A61Q 19/10; A61K 8/41; A61K 8/922; A61K 8/55; A61B 5/14546; A61B 5/15003; A61B 5/150206; A61K 2800/28

(57) ABSTRACT

This disclosed method improves the accuracy of testing blood for the levels of contaminant, such as lead, cadmium and mercury, in individuals. The method comprises cleaning the area where the skin will be penetrated to obtain the blood sample to remove the contaminant to be measured in the blood. The cleansing is accomplished with a cleanser formulated to remove the contaminant to be measured in the blood from the surface of the skin, the pores, sweat ducts, hair follicles and sebaceous gland ducts. The method reduces contamination of the blood sample by contaminants on, and/or in the portion of the skin through which the blood sample is drawn. A premoistened wipe can also be used that mobilizes heavy metals from the skin surface, the skin pores, sweat ducts, hair follicles and sebaceous gland ducts, and is formed with a wipe substrate material selected for its affinity to bind the toxic materials.

### CROSS-REFERENCE TO RELATED APPLICATIONS

This application is a continuation of U.S. patent application Ser. No. 11/487,183, filed Jul. 13, 2006, which claims priority under 35 U.S.C. §119(e) from U.S. Provisional Patent Application Ser. No. 60/699,286, filed on Jul. 14, 2005, the entirety of which is expressly incorporated herein by reference.

### FIELD OF THE INVENTION

The present invention relates to blood sampling methods, and more specifically to an improvement in the method of collecting a blood sample for subsequent analysis for contaminants including heavy metals, trace metals or other materials in the blood that significantly reduces the contamination of the blood sample during collection thereby improving the accuracy of the results.

### BACKGROUND OF THE INVENTION

I. Reasons for testing Blood for Lead, Cadmium, Mercury and Trace Metals

The Centers for Disease Control and Prevention (CDC) and the Occupational Safety and Health Administration (OSHA) both recognize testing the blood levels of individuals for lead and cadmium is the most economical and reliable means for determining the overall exposure to these metals and assessing the health related effects based on these levels. Measuring low levels of mercury in blood may also be desirable for at risk individuals. As more becomes known about the toxic effects of other metals and with improvements to analytical methods, it may be at some point desirable to test the blood for other toxic metals or contaminants. At present, the CDC recommends a maximum blood lead level for children under the age of sixteen years old and pregnant or breast feeding women of 10 micrograms per deciliter of whole blood (μg/dL). They recommend a maximum blood lead level of 25 μg/dL for all other persons. OSHA regulations currently require the removal of individuals from further exposure to lead at a level of lead in blood of 50 μg/dL for lead exposed workers (20 CFR 1910.1025 and 1926.062). OSHA has expressed a strong interest in reducing the 50 μg/dL blood lead limit in the near future. The OSHA cadmium regulation at 29 CFR 1926.1027 requires cadmium exposed workers be removed from further cadmium exposure if their blood cadmium level exceeds 5 micrograms per liter of whole blood (μg/lwb).

The CDC and the American Academy of Pediatrics (AAP) have recommended that all at risk children under the age of six (6) have a screening test to determine their blood lead level, so that Public Health Professionals can intervene for children with elevated blood lead levels. Additionally, blood lead testing of children enrolled in all state Medicaid programs is federally mandated by the Centers for Medicare and Medicaid Services (CMS).

Intervention in occupationally exposed individuals by medical removal from sources of exposure to lead or other heavy metals is a potentially large cost for companies in the lead and cadmium processing industries. In the public health sector, the cost to the government can also be large with respect to the public health staff time that is devoted to investigating an elevated blood lead level. Falsely elevated results also create unnecessary anxiety for the individual, parents and family.

An atomic element, which is beneficial in amounts smaller than 0.01% of the mass of the organism, is called a trace element. Metal ions such as sodium, potassium, magnesium, and calcium are essential trace metals required to sustain life. In addition, six other metals are also essential for optimal growth, development, and reproduction. These include manganese, iron, cobalt, copper, zinc, and molybdenum. These six metals are all transition metals. However, all essential trace metals become toxic at excessive levels.

All of these trace metals are measured in blood by withdrawing a blood sample through the skin for chemical analysis. In addition to sampling the blood of an individual for testing for these types of contaminants, there are many substances present in the blood where there is a need to acquire a sample to measure the concentration. Some examples of these other tests are: glucose, hemoglobin, henotocrit, creatinine, blood gases and drugs. Each of these blood samples are subject to contamination during the process of obtaining the sample. More particularly, when the blood sample is collected, any contaminants, e.g., lead, cadmium or other trace metal, present on or in the skin can contaminate the blood sample, causing an unrepresentative increase in the metal concentration of the blood sample. These extra metals did not reside in the blood, and are actually surface deposited metals from external environmental origins or excreted from the sweat glands and the sebaceous glands or metals dissolved in the sweat as well as trace metals present throughout the entire depth of the skin layer. In the case of blood tests for trace metals, the skin contamination is an uncontrolled variable in the sampling process that affects the results. This currently uncontrolled variable inserts a degree of randomness into the results of routine blood tests for all metals in the blood.

Two types of blood are sampled for metals analysis: capillary blood and venous blood. Arterial blood is rarely used for screening tests or medical diagnosis and monitoring. In order to acquire a sample of blood for metals analysis, the skin must be penetrated, and the blood must flow through the skin layer to reach the surface. The depth of skin penetration during sampling ranges from 1.5 mm to 3.5 mm. The wound from the incision ranges from 0.375 mm² (lancet) to 0.7 mm² (needle). In the case of venous blood samples, the need to eliminate surface contamination prior to insertion of the needle is ignored by standard protocols. In the case of capillary blood samples, the necessity to clean the surface of the skin at and around the stick site is addressed by sampling protocols, but does not address the need to use skin cleaners that are highly effective at removing the specific contaminants that will affect the analytical result. It is incorrectly assumed by these protocols that all soaps and skin cleaners have a high efficiency for the removal of the specific contaminant of interest. Further, in addition to not adequately addressing the issue of contamination on the surface of the skin, there is the issue of contamination within the skin through which the blood sample is obtained that needs to be addressed.

### II. Lead and Cadmium Absorption

Inorganic forms of lead and cadmium enter the body primarily by inhalation and ingestion. Lead on the skin can readily be transferred to the mouth, where it can be ingested or inhaled, or to the nose for inhalation by hand to face activities of an exposed individual. Occupational and pediatric experience has shown that the hand to face pathway is a significant source of exposure by both inhalation and ingestion. Water soluble, inorganic forms of lead, as well as organo-lead compounds can be absorbed through the skin barrier, resulting in not only surface contamination, but also subsurface contamination. Water soluble metal salts can migrate through intact, healthy skin into the extracellular fluids (ecf) and the lymph system. Mercury compounds and elemental mercury are absorbed by inhalation, ingestion and skin absorption. Some lipid soluble organic complexes, such as tetraethyllead can be absorbed through the skin.

When lead or cadmium is inhaled, a portion of it is absorbed and transferred to the blood. The balance is rejected by the body and removed by lung clearance mechanisms before it is absorbed. A portion of ingested lead and cadmium are also absorbed and transferred to the blood, with the balance passing through the intestines and excreted. In the example of lead, the body can place the lead into storage or excrete it. The body has a variety of means it utilizes to eliminate lead, cadmium and mercury. For example, lead is stored in all types of body tissues, bones, teeth and organs where it causes damage to these systems. This lead storage is not static, however, lead that has been placed in storage by the body, remains in “circulation” due to the exchange between lead atoms in the blood and lead atoms in the bones for example. Lead in soft bone exchanges at a particularly fast rate. Lead in hard bones and teeth exchanges with lead in the blood at a very slow rate. Lead in soft tissues exchanges at a moderate rate.

The body excretes lead by every mechanism available to it. Lead is removed by the kidneys and is excreted in urine. Unabsorbed, ingested lead is excreted in feces. Lead is also excreted in saliva as spittle. Mucous discharges generated by lung clearance mechanisms, including coughing also excrete lead. Lead is also excreted in hair, fingernails, dead skin cells and sweat. The half-life of adult blood lead has been variously estimated at 25 to 36 days. The half life of pediatric blood lead appears to be a matter of some debate.

It is also well known that both the beneficial and toxic trace elements are excreted in sweat, and that excessive sweating can deplete the body's levels of the beneficial, essential trace elements. Both the skin surface and the subsurface can have high concentrations of both toxic metals and beneficial trace metals originating from sweat as a completely separate source of contamination apart from environmental sources.

A. Lead in and on the Skin

Lead also resides on and in the skin of exposed individuals. Lead particulate, as well as all of the other metals and arsenic (a metalloid, it sometimes behaves as a metal and other times as a non-metal, and is in a classification unto itself) form extremely small particles. Larger particles, such as those originating from paint dust abrade easily into finer and finer particles, with a substantial portion of this dust less than 10 microns in diameter. Lead and cadmium particulates from industrial sources are also extremely fine. Production specifications for both lead oxide and cadmium oxide are typically 100% less than 3 microns. Metal dust is also formed by evaporation from a pool of molten metal and the particles are typically 20% less than 0.5 micron in diameter.

The essential trace transition elements also form extremely fine particles in the case of industrial processes and evaporation of molten metal, as well as precipitation from solution. The environment provides a source of metal contamination on the exterior surface of the skin; by deposition of metal particles from the environment. In addition, metals can dissolve in sweat and migrate through the skin via the sweat ducts into the skin and extracellular spaces between the skin cells.

As a result of the small size of the particles and metal ions present, and the large variety of attractive forces binding lead (or other metals) to the skin, lead dust on the skin will also reside in the pores. If the skin is treated as a box, it has a surface area of about 2 square meters in the male adult. However, when all of the skin pores, interior porosity of the dead, desiccated epidermal skin cells and other skin structures open to the surface are taken into account, the actual surface area of the skin is significantly greater than this. Some of the attractive forces include electrostatic forces (most metal oxides accumulate and hold a large static charge), mechanical (entrapment occurs when the particle corresponds to the diameter of the skin pore or the pore of a desiccated skin cell), and cross linked attractions to the water that hydrates the skin; as well as adhesion of the particle by oils on the skin. Adhesion of metallic, metal oxide and metal salt particles is increased by natural skin oils secreted by the sebaceous glands, as wells as commonly used skin lotions and oils. Metals are difficult to disperse in water, they have high densities and the high weight density of the individual particles makes them difficult to disperse and float in water unless they are dissolved. In addition, elemental metal, metal oxide and metal salt particles tend to be both sticky and repel water (difficult to wet). Oxides of lead, cadmium and other metals also will hold a static charge, providing an additional means of binding these materials to the skin.

B. Structure of the Skin

As shown in FIG. 1, the skin is structured to prevent loss of essential body fluids, and to protect the body against the entry of toxic environmental chemicals. The overlapping cells and intercellular lipids of the outer stratum corneum layer, makes diffusion of water into the environment very difficult. The skin also provides part of the natural resistance of the body against invasion by micro-organisms. The dryness and constant desquamation of the skin, the normal flora of the skin, the fatty acids of sebum and lactic acid of sweat, all represent natural defense mechanisms against invasion by micro-organisms.

Skin protects body tissues against injuries and helps regulate body temperature. The surface of the skin contains a very large amount of surface area when viewed on the micron scale. At the micron scale, an electron microscope view of the topmost layer of the epidermis closely resembles a flaky puff pastry in appearance. The surface of the skin is made up of dead, desiccated, stratified epidermal cells that are highly porous. This is called the horny layer and the dead skin cells are a type of keratin. The skin continuously renews itself, new cells are formed in the basal layer and the older cells die and are pushed to the surface by newer ones to protect the live, healthy skin cells below.

The interior surface area of each of the individual skin cells can easily exceed the external surface area of these cells, much like the porous interior of activated charcoal particles. There are large gaps between the individual keratin flakes of the horny layer. Dermal fingerprint ridges are ˜500 microns wide and up to 50 microns deep. Hair follicle shafts are 50-100 microns in diameter. Scent secreting apocrine glands are ˜200 microns in diameter, while eccrine (sweat) glands are ˜20 microns wide. The skin is almost always in constant motion, at size scales up to ˜1,000 microns, folding and unfolding, stretching and tightening, twisting and curing, with normally distant surfaces being brought into and out of contact continuously.

The skin is frequently covered with dirt, grease, cooking oils, fats and sebaceous gland oils which can be tens to hundreds of microns thick. The skin can also pour out as much as 2 liters per hour of perspiration. This perspiration contains all of the beneficial and toxic trace elements in the body, and these permeate the desiccated keratin cells, the pores and the spaces between the cells and the deposited solids adhere to all of the available surfaces exposed to the sweat.

While the skin over most of the body is relatively smooth when viewed at the macro level, friction ridges are found on the digits, palms and soles. They are called friction ridges because their function is to assist in our ability to grasp and hold onto objects. These ridges vary in length and width, branch off end suddenly and, for the most part, form into distinct patterns. There are approximately 4.25 ridge “units” per square mm of friction skin. Each ridge “unit” corresponds to one primary epidermal ridge (glandular fold) formed directly beneath each pore opening. Pore openings are present along the surface of the friction ridges and the valleys between them.

C. Sweat Glands

There is on average 1 sweat gland per square millimeter on the surface of the human body for an average person and they are quite evenly distributed over the entire surface. In the early years of life, they produce little sweat until the child's thermoregulatory system matures and they are able to regulate their body temperature in this way. However, the sweat glands and skin pores are present, every though they produce little sweat. Sweat glands secrete mostly water, sodium chloride (salt), urea, ammonia, and uric acid. Urea, ammonia, and uric acid are waste products of protein metabolism. These waste products are toxic to the body. There are two types of sweat glands: Eccrine and Apocrine. Most of the sweat glands are of the Eccrine type. Sweat glands of the Apocrine type discharge into hair follicles.

Sweat glands are coiled, tubular glands. Their ducts open at the skin's surface, similar to the opening of a hair follicle. The glands secrete sweat for three main purposes: to moisten skin, to excrete waste, and to regulate body temperature. Once secreted onto the surface of the skin, the sweat evaporates, cooling the surface and depositing the solids. The porous structure of the desiccated surface cells greatly increase the surface area for evaporation, and the pores open and close as needed to regulate skin temperature and evaporation rate.

A second type of sweat is present in the armpit, nipple, and anal regions. These glands, the Apocrine glands, open into a hair follicle, rather than directly onto the skin's surface. These glands, the Apocrine glands, produce a thick, sticky secretion.

Lead, cadmium and the other metals occur in sweat as part of the body's excretion mechanism. Water soluble forms of lead and cadmium on the skin can migrate through the sweat glands and the hair follicles, and circulate in the lymph system. Excreted metals are found in the transcellular water and extracellular fluid that form a component of sweat. From here it can be secreted at any point on the body in sweat. These compounds also reside in the pores of the skin, the extra cellular fluids, the spaces between skin cells, and the interior and exterior of the dead (flaking) skin cells.

Since all human beings are exposed to both the toxic metals and the trace elements, levels of the metal exposed reside on and in the surface of the skin. Skin levels correlate strongly with blood levels. Askin, D P and Volkmann, M in “Effect of Personal Hygiene on Blood Lead Levels of Workers at a Lead Processing Facility”, Amer. Ind. Hyg, Assoc. J, (1997), 752-753 used D-Wipe® Towels to measure the amount of lead on the right hand of workers and found a highly significant correlation between the quantity of lead recovered from the hand and the worker's blood lead level. (Positive correlation coefficient was 0.61 and p<0.002).

According to Stauber, et al, in Percutaneous Absorption of Inorganic Lead Compounds” the Science of the Total Environment 145 (1994) 55-70, and his predecessors in the field, lead on the skin behaves as follows:
1. Water soluble forms of lead, (such as lead nitrate and lead acetate [water soluble salts]), as well as elemental lead are absorbed through the skin via the sweat ducts and hair follicles on the skin.
2. Sweat secretion of lead varies depending on skin hydration, occlusion, physical activity and atmospheric conditions.
3. Lead absorbed through the skin does not pass into the blood or urine at significant levels.
4. Lead is soluble in synthetic sweat at a level of 40 mg/L (lead oxide) to 56 mg/L (lead metal) and in sauna sweat at 6 mg/L (lead metal) to 20 mg/l (lead oxide).

According to Lilly, et al, “The Use of Sweat to Monitor Lead Absorption through the Skin” the Science of Total Environment, 76: 267-278; Florence at al, 1988, “Skin absorption of lead”, Lancet, 16: 157-158 and Omokhodian and Howard, “Sweat Lead Levels in Persons with High Blood Lead Levels: Lead in Sweat of Lead Workers in the Tropics”, the Science of Total Environment, 103: 123-128.

Lilly at al proposed an absorption mechanism whereby environmental sources of lead dissolve in sweat and the lead ions diffuse rapidly through the filled sweat ducts, followed by a slower diffusion through the stratum corneum.

Omokhodion reported sweat lead levels in lead exposed workers with blood lead levels between 13 and 36 μg/dL ranged from 72 to 256 μg/liter. “Their sweat lead levels were higher than their urinary lead levels in all cases and even higher than blood lead levels in some workers.”

“Nevertheless, it can be concluded that, in occupationally exposed persons, sweat lead losses are derived from body stores and the local excretion of lead absorbed from the skin”.

Florence, et al, in “Skin absorption of lead”, Lancet, 2 (1988), 157-158, reports sweat lead levels in battery workers as high as 800 μg/liter, in men with blood lead levels of 30 to 40 μg/dL. Lead in the sweat of nine lead workers with blood lead levels of between 18.6 and 95.2 μg/dL ranged from 71 to 17,700 μg/liter (17.7 mg/L).

Daily sweat excretion varies from 0.05-4.0 liters, depending on temperature, humidity, exercise and acclimatization. Sweat volumes can be as high as 2 liters per hour.

Stauber and Florence in “The determination of trace metals in sweat by anodic stripping voltammetry”, Sci of Total Env, 60: (1987) 263-271 state: “The most likely source of trace elements in sweat is blood serum; labile metals dissociate from proteins under the influence of the concentration gradient existing across the blood capillaries, and diffuse through the capillary walls into the sweat glands.”

As an example, an individual with a blood lead level of 30 μg/dL, a sweat lead level of 250 μg/liter, discharging 1 liter of sweat per day onto the exterior skin surface of 2 m² will deposit 125 μg of lead/m² over the surface of their skin. This equals 12.5 nanograms of lead per square cm, or 0.125 nanograms per sq mm over the surface of their skin each day. However, in order to discharge 1 liter of sweat onto the surface, more is produced that does not reach the surface. Some of the metals content of this diffuses into the skin layer. These metals accumulate and the quantity present increases from day to day by the quantity that is not discharged to the surface or removed by washing or exfoliation.

### III. Blood Sampling

When the blood sample is collected, any lead, cadmium or other trace metal present on or in the skin can contaminate the blood sample, causing an unrepresentative increase in the metal concentration in the blood sample. These metal contaminants in the sample did not originate in the blood, they originated as surface deposits from environmental sources or they originated from metals excreted from the sweat and sebaceous glands, or as trace metals present throughout the entire depth of the skin layer.

In order to collect a blood sample, the skin must be penetrated. There are two conventional types of blood samples collected for determining metal concentration in blood. They are capillary blood and venous blood. Capillary blood is used principally for screening and in the event of an elevated capillary result the CDC recommeds a follow up confirmation test be done. Venous samples can be and are used for screening purposes, and in the event of an elevated venous screening result, the CDC does not recommend a confirmatory test. Due to concerns over the accuracy of capillary blood lead testing, the CDC recommends that all elevated capillary blood lead test results be confirmed by a subsequent blood lead test. A venous blood lead test is considered to be more accurate because it has been viewed as less susceptible to contamination See: http://www.dc.gov/nceh/lead/guide/1997/pdf/c1.pdf )

Under OSHA rules, monitoring of worker blood and cadmium levels are almost always done with a venous sample and in the event of an elevated result, whether venous or capillary, a follow up confirmation test is required within 7 days.

The Centers for Medicare & Medicaid Services (CMS) regulates all laboratory testing (except research) performed on humans in the US through the Clinical Laboratory Improvement Amendments (CLIA). The Division of Laboratory Services, within the Survey and Certification Group, under the Center for Medicaid and State Operations (CMSO) has the responsibility for implementing the CLIA Program.

Clinical Laboratories must be licensed under CLIA to provide analysis of lead in blood and for most of the other metals. The majority of blood lead samples are analyzed by one of these methods: Anodic Stripping Voltammetry (ASV), Graphite Furnace Atomic Absorption Spectroscopy (GFAAS), Inductively Coupled Mass Spectroscopy (ICMS) and LeadCare® ASV.

In the US blood lead levels are typically reported in units of micrograms of lead per deciliter of whole blood (μg/dL). Other reporting units in use include: micrograms of lead per 100 grams of whole blood (μg/100 g), micromoles of lead per liter of whole blood (μmols/L). Blood cadmium levels are typically reported as micrograms of cadmium per liter of whole blood (μg/lwb). Conversion factors can be used to convert between these different units. All of the trace metals are typically reported in these same units.

A. Definition of Capillary Blood Lead Specimen Accuracy

Capillary blood lead testing accuracy has been measured by various means in published studies. We use the following parameters for defining capillary blood testing accuracy that are defendable, meaningful, and most importantly, useful to those involved in actual blood lead testing activities. We define an accurate capillary blood lead test as any test meeting one or more of the following criteria:

1. Any capillary test in which the result is <10 μg/dl.

2. Any capillary test ≧10 μg/dL for which the result of a subsequent venous confirmatory test performed within 90 or fewer days of the capillary test is ≧the result of the capillary test.

3. Any capillary test ≧10 μg/dL in which the result of a subsequent venous confirmatory test performed in 90 or fewer days of the capillary test is no more than 4 μg/dL less than the result of the capillary test.

Follow up confirmatory tests are almost always done with a venous sample. A venous blood lead test is considered to be more accurate because it has been viewed as less susceptible to contamination by lead. (See: http://www.cdc.gov/nceh/lead/guide/1997/df/c1.pdf)

Our reasons for defining accuracy in this manner are as follows:

For Criterion #1: The CDC defines an elevated blood lead specimen as any specimen in which the lead content is ≧10 μg/dL. Therefore, if the result of a capillary blood lead test is <10 μg/dL, it is reasonable to assume that either.
a. No pre-analytic contamination has occurred, and the result is accurate.
b. Or, if pro-analytic contamination has occurred, it is of no clinical significance since the result is below the CDC-defined elevated level of 10 μg/dL.

For Criterion #2: The maximum interval, recommended by the CDC, between the detection of an elevated blood lead level by a capillary test, and the collection of a confirmatory venous specimen is 90 days. Blood lead half-life can have a significant impact on the correlation between determined capillary blood lead levels and those of subsequent venous testing. For example, it is theoretically possible for a capillary BLL of 18 μg/dL and a venous BLL of 9 μg/dL determined 28 days later to both be accurate.

Therefore, the use of data for only those capillary tests for which a confirmatory venous test was performed within 90 days is reasonable in that it represents the universe of testing performed in actual practice.

The primary concern over the use of any capillary blood lead test is that, due to specimen contamination, it may produce a falsely-elevated result (a result that is higher than the true blood lead level). A capillary blood lead result is defined as “falsely-elevated” whenever the capillary result is ≧10 μg/dL and a venous confirmatory test performed within 90 days of the capillary test is <10 μg/dL.

Therefore, it is reasonable to assume that if the result of a confirmatory venous test is higher than the result of the initial capillary test, that the capillary test was accurate and not subject to pro-analytic contamination, and that the higher venous test result reflects the fact that the patient's blood lead level increased during the period of time between the two tests due to continued exposure to lead.

For Criterion #3: CLIA-approved blood lead proficiency testing programs typically consider a capillary result that is within + or −4 μg/dL of a target value established by venous testing to be of acceptable accuracy.

Additionally, it is generally acknowledged that the accuracy achieved by various laboratories may deviate from the true blood lead level by up to 4 μg/dL.

Given these parameters for acceptable accuracy and methodological precision, adopted by CLIA and the CDC it is reasonable in this context to define a capillary blood lead test that is no more than 4 μg/dL greater than the subsequent venous test result as accurate and uncontaminated.

However, it is essential that any blood sampling be done in a manner that reduces as much as possible the potential for a sample that is contaminated and that produces a falsely-elevated and inaccurate test result. This situation is made all the more important by the relatively small amounts of metals or other contaminants that will elevate the test results to an unacceptable level, requiring additional testing and costs for that testing. For example, Table 1 shows the quantity of lead contamination in a blood sample that would raise the analytical result by 10%, based on an actual blood lead level of 20 μg/dL.

TABLE 1
Quantity of Lead Contamination to raise
Blood Lead Sample Value by 10%
Based on Blood Lead Level of 20 micrograms per deciliter

| | | | | Weight of Lead |
|---|---|---|---|---|
| | Sample | Total Lead | Total Lead | Contamination |
| Sample | Volume | in Sample | in Sample | to raise value |
| Method | (mL) | (μg) | (ng) | by 10%, nanograms |
| Filter Paper | 0.05 | 0.01 | 10.0 | 1.0 |
| or Capillary Tube | | | | |
| Lead Care ® | 0.05 | 0.01 | 10.0 | 1.0 |
| Analyzer | | | | |
| Capillary Tube | 0.25 | 0.05 | 50.0 | 5.0 |
| Venous tube | 2.00 | 0.40 | 400.0 | 40.0 |
| Venous Tube | 20.00 | 4.00 | 4,000.0 | 400.0 |

B. Capillary Blood Sampling

For capillary blood samples (Filter Paper [FP] or Capillary Tube [CT]), a retractable lancet is used to pierce the skin, typically on the side of the 3^{rd} or 4^{th} fingertip, the heel, the toe or the earlobe. The lancet penetrates to a depth of about 2.2 mm, and a drop of blood flows through the skin opening where it forms a drop of blood on the skin surface. For capillary tubes, several of these drops of blood, totaling approximately 250 microliters (uL), are drawn into a capillary tube containing an anticoagulant, then sealed, mixed and transported to the laboratory for analysis.

Alternately two drops of blood, approximately 30 to 50 microliters (μl) are sequentially deposited onto different locations on a piece of filter paper, dried, sealed and then transported to the laboratory for analysis. (The difference in volume is a result of different laboratories having slightly different analytical procedures.)

Typical blood sample volumes for blood lead and cadmium measurements vary from laboratory to laboratory, and are specified by the laboratory according to their different analytical procedures, but typically are as follows:

TABLE 2
Representative Capillary Blood Sample Volumes

| | |
|---|---|
| Filter Paper [FP]: | 30 to 50 μl of whole blood (Stanton procedure) |
| Capillary Tube [CT]: | 50 μl of whole blood (Sinclair procedure) |
| | 50 μl of whole blood (LeadCare ® ASV |
| | procedure) |
| | 250 μl of whole blood (Missouri procedure) |

C. Sources of Lead Contamination affecting Capillary Blood Lead Samples

Lead is present on the skin and in the skin. The amount of lead present typically increases with increasing blood lead level, but is not a predictor of the blood lead level.

For a capillary blood sample, the lancet wound size of 1.5 mm by 0.25 mm (0.375 mm²), would cut through 4 skin friction ridges, with a total disrupted surface area of 0.70 mm² (in the case of a finger, toe or heel sample location).

Lead of environmental origin can be present on the skin in the form of finely divided particles. Lead has a specific gravity of 11.34 grams per cubic centimeter=0.01134 nanograms per cubic micron. Thus a single, cubic lead particle with dimensions of 1 micron weighs 0.01134 nanograms and a 10 micron particle weighs 11.34 nanograms.

On the upper, 3 dimensional surface of the stratum corneum, there would be room for 50,000 lead particles each measuring 10 micron on a side, one layer thick. Thus, there is sufficient probability for an individual exposed to environmental sources of lead to have the equivalent of one or more lead particles present in the sample area. A single micron lead particle is sufficient to raise the value of a 50 μl blood sample by 110% at the 20 μg/dL level increasing the analytical result to 41 μg/dL. At higher environmental exposures to lead, the greater the probability that lead will be present in the sample area, and that the amount of lead present will be significant with respect to the blood lead sample.

There is also a high probability of sweat lead to be present. For the case of the individual with a blood lead level of 30 μg/dL, producing 1 liter of sweat per day containing 250 μg of lead per liter, this adds an additional 0.1 nanograms of lead to the 0.7 mm² sample area. Since this lead can accumulate from day to day, and build up, it will easily exceed 1 nanogram over the wound area, enough to raise the sample value by 10%.

Even in the absence of any external environmental source of lead contamination, the drop of capillary blood must travel through about 2.2 mm of the skin layer to reach the surface. The surface area of the cylinder totals 8 sq mm of additional surface (if we treat the walls as smooth, sheer surfaces). The capillary blood can be exposed to lead contamination as it flows through this opening. At a surface loading of 0.125 nanograms of lead per sq mm for this surface, this provides the potential for an additional 1 nanogram of lead contamination (0.125 ng/sq mm*8 sq mm).

After the skin is penetrated, the blood flows to the surface, pools on the skin and forms a droplet. When the drop of capillary blood pools on the surface of the skin, it forms a drop approximately 2 to 4 mm in diameter. For a 4 mm droplet, this covers an area of 12.5 square mm. In this example, the drop of blood is potentially exposed to an additional 1.6 nanograms of lead.

In the absence of any external skin contamination of the skin, these 2 subsurface sources of contamination together equal 1+1.6=2.6 nanograms of lead. As illustrated in Table 2, this amount of lead is sufficient to increase a 50 μl capillary blood sample by 26%, or a 250 μl sample by 5.2%.

In addition to the potential for environmental lead and sweat lead to contaminate the blood sample, another source of lead can contaminate the blood sample. Anytime the skin is cut, as in this case of a lancet penetrating the skin, skin fragments are dislodged, or cut away and pushed through the skin. These skin fragments can also be contaminated. Since the capillary blood is under pressure, this skin debris is pushed out into the sample and is present in the blood sample. When lead is present in the skin, these skin fragments contain the metal contaminant and are incorporated into the blood sample.

In the absence of any external environmental sources of lead our analysis of the skin as a two dimensional surface, shows a high probability of contamination at the 5% level. Now, to this surface area, we must add the additional surface area present inside, under and between the desiccated surface cells, the interior of the sweat glands, the skin pores and other internal surfaces. These surfaces increase the surface area of skin that is in contact with the blood sample by thousands of times compared to the 2 dimensional area of the incision.

The word adsorb is important here. Lead and other metals on the skin are adsorbed, attaching themselves to all of these surfaces by chemical, physical and mechanical forces. The extensive interior and exterior surface area of the desiccated skin cells, as well as the pores and sweat ducts give the metal contaminant countless attachment sites. When contaminants come into contact with the skin, they not only attach to the relatively limited exterior, upper surface, the also attach to the interior surfaces. These interior surfaces accumulate metals from day to day. This is true for both environmental particles of lead, and environmental lead that has dissolved in the sweat and has migrated into the skin, but also lead from body stores excreted in sweat, which thoroughly penetrates the porous skin cells and all of the other open structures in the skin.

For individuals with dry, chapped, damaged skin or otherwise in poor condition, the surface area of the skin can be again 100's of times greater than the surface area of an individual with smooth, healthy skin. This is due both to the porosity of top layers of dead skin cells, but also to the peaks and valleys formed by the dead, desiccated skin cells creating a surface resembling the surface of a pine cone.

In summary, even in the absence of any environmental lead, this potential contamination is sufficient to raise a 20 μg/dL blood lead level in a capillary blood sample by more than 5%. The presence of even a single 10 micron lead particle is sufficient to raise the blood lead level by 110%.

In “Diagnostic Testing Unwarranted for Children with Blood Lead 10 to 14 ug/dL, Sargent, J D, Dalton, M, and Klein R Z, Pediatrics, Vol. 103 No. 4 April, 1999, p. e51, the results of capillary blood screening and venous blood lead screening were compared with follow up venous confirmation tests collected within 90 days for thousands of children in Massachusetts and Rhode Island. The error rate for venous screening samples was 42% and the capillary screening error rate was 77%. “Higher capillary screening misclassification error rates among capillary screening samples is probably attributable to positive bias in the measurement of capillary screening samples resulting from finger skin contamination with lead.”

D. Venous Blood Sampling

Capillary blood samples are typically collected to screen large populations for potential lead or cadmium problems. They also can be used to screen for the full range of metals in blood, as well as other tests, for example, hemoglobin. Venous samples are normally used for medical diagnosis and are usually used to confirm elevated capillary results for toxins, and low results for beneficial metals, even in the pediatric population. Venous samples are used almost exclusively for occupationally exposed individuals due to the concerns of potential contamination of capillary samples. It is very common for individuals having a venous sample collected to assess their blood lead level to have a high probability of lead on the exterior skin surface. As discussed previously, the concentration of lead in sweat increases with increasing blood lead. The frequency of lead particles on the skin is greatly increased for exposed individuals and for individuals who have a blood lead level in the range of concern.

In a venous blood sample method the needle is inserted through the skin into a vein, typically located just below the inside of the elbow. The needle is inserted at a 15 to 30 degree angle and penetrates the skin to the depth necessary to enter the target vein near the surface of the skin, approximately 3.2 mm, as shown in FIG. 2. Blood pressure propels the blood into an evacuated container containing an anticoagulant, and the sealed blood sample container is subsequently transported to a laboratory for metals analysis.

TABLE 3
Representative Venous Blood Sample Volumes

| | |
|---|---|
| Venous Tube [VT]: | 2-5 mL, minimum of 1 mL (Missouri procedure) |
| | 20 mL (Sinclair procedure) |

E. Sources of Lead Contamination Affecting Venous Blood Lead Samples

A core of skin is cut out and due to the pressure of the blood in the vein is pushed into the collection tube. During the cutting process, skin cells are disrupted and fragments of skin also enter into the collection vial after the skin core. So in this instance, we have potential contamination from material deposited on the skin from environmental and sweat origins, as well as the solid residues remaining after the sweat has evaporated as well as the lead present in the skin layers and dead stratum corneum layer.

For a venous sample, collected with a 21 gauge needle (measuring about 0.93 mm inside diameter), the needle cuts an opening in the skin covering 1,060,000 sq microns, and the diameter of the care removed from the skin is 0.93 mm in diameter=700,000 square microns, or 0.7 sq mm. The total surface area of the exterior skin surface disrupted, taking into account the ridges on the skin, is almost 1,500,000 square microns, more than sufficient space for the 40 ng of lead particles (3.5 particles 10 micron in size) to contaminate a 2 mL sample and for 400 ng of lead (35 particles 10 microns in size) to contaminate a 20 mL sample. These 35 lead particles represent 0.023% of the 1.5 sq mm exterior surface area of the skin disrupted to obtain a blood sample.

This core of skin has a calculated weight of 1.3 milligrams, or 1,300,000 nanograms. If this core of skin contains 0.01% by weight lead, that would deposit 130 nanograms of lead into the sample vial.

This core of skin will contain a skin pore, a sweat duct, and occasionally a hair follicle and/or a sebaceous gland. There is about 1 sweat gland and one skin pore for every square mm of skin. As the needle creates a wound covering 1.06 square mm, it is highly probable that a sweat gland and a pore will be incorporated into the skin sample, or at the very least disrupted and adding its contents to the sample. Thus, this core of skin will also contain at least one, and often more of a sweat gland, pore, hair follicle or a sebaceous gland. Skin pores hold lead in exposed individuals. Sweat ducts and hair follicles are excretion paths for lead.

But not all of the disrupted skin will travel in or with the core of skin. The cut also generates skin fragments that mix into the blood sample for the entire duration of the sample collection. These skin fragments originate from the incision, as well as scraping of the walls of the incision. The surface area of the cutting edge approximates 382,000 sq microns, or 0.38 sq mm. Whenever any lead particles or lead ions are located in this area, they are transferred to the cutting edge of the needle and they can be washed into the sample by the blood flow. The 35 micron sized particles required to contaminate the sample for a 10% increase will obviously fit into this area.

The experimental results discussed in the experimental section show that on venous blood lead samples collected on the same day from the same individuals, using different stick site preparation methods indicate that up to 76% of the venous samples had some detectable level of lead contamination.

Due to the skin contamination that is present in lead exposed individuals, (100% of the world population is exposed to lead and all of the other metals in commercial use) venous samples also tend to produce falsely-elevated results, and thus, the 10% maximum falsely elevated value cited by the NY State Department of Health Wadsworth laboratory (in Capillary Blood Sampling Protocol See: http://www.leadposion.net/screen/capillary.htm) may be inaccurate since it is based on venous samples collected with the standard stick site preparation protocol. Our experimental data with lead exposed individuals shows that venous blood lead samples may be contaminated up to 76% of the time when they are collected with the standard stick site preparation protocol.

We have observed that in the case of venous samples good practice may include not using the first vial containing the core of skin for metal analysis. The first vial can be used for other tests, (e.g. creatinine), or discarded, and a second vial tested for metals. Our tests indicate that when the second vial of venous blood is tested from a single stick site, the blood lead level is typically 5 to 10% less than the lead level in the first vial. Removing more blood from a healthy individual than is absolutely necessary does not conform to good, accepted medical practice. It also increases the quantity of biological waste generated and increases testing costs. The additional lead comes from the lead contamination on and in the core of skin deposited in the vial.

### IV. Prior Art—Soap and Water, Skin Sealants or Barriers, Acid Wash and Alcohol

The development of the screening methodologies and analytical procedures to measure capillary blood samples date back to the 1950's, and the potential for skin contamination of capillary samples was identified early in the development of these methods. Over the years, various methods have been used by researchers to control or eliminate contamination of the blood when collecting a capillary blood sample. Some of the contamination control steps currently utilized during sample collection to attempt to assure the maximum accuracy of a blood sample include:
1. Wash the area around the stick site with soap and water.
2. Clean the stick site with an alcohol prep pad.
3. Assure the sampling area is clean, including work surfaces, air, gloves and clothing, with respect to the analyte of interest.
4. Assure all of the sample containers, needles, lancets, filter paper and all other materials that will or may come into contact with the blood sample are as free of the analyte of interest as is economically or technically feasible.
5. In the special case of filter paper sampling methods, the air used to dry the filter paper is kept free of the analyte of interest, or the paper protected in another manner during the drying step.
6. At every step of the laboratory analysis, strict contamination control steps are implemented and maintained.

Most, but not all, of the capillary blood sampling protocols published by the CDC and the various State Lead programs include the step to wash the sample area with “soap and water”. (See:http://www.cdc.gov/nceh/lead/Publications/books/plpyc/appendix.htm (CDC) and http://www.dhss.mo.gov/Lead®/Section2.doc (Missouri))

The CDC protocol states: “The child's hands should be thoroughly washed with soap and then dried with a clean, low lint towel. If water is unavailable, foam soaps can be used without water.”

The Missouri protocol states: “REGARDING HAND WASHING: It is important to wash the child's hands including front and back, in between the fingers, around the nails, and underneath the nails to get a correct test.
4. Wet the child's hands apply liquid soap and lather well. (You may want to use SOFT brush to help clean the nail area.) Rinse hands well letting the water nm from the wrist area into the sink. Dry with a paper towel and then get a clean paper towel to wrap around the hand. Keep the paper towel over the hand. The parent/caregiver can assist you by holding the towel in place.”

Researchers have also investigated additional steps to reduce blood sample contamination introduced from the skin surface during sample collection. These methods include washing or rubbing the sampling site with dilute nitric acid, vinegar and/or the use of barrier sprays, including silicone, rubberized adhesive wound dressings and collodion sprays. The use of these types of contamination preventative measures have been investigated or cited in many blood sampling research studies for both capillary and venous sampling procedures, such as the following:
De Silva P E, Donnan M B. Blood lead levels in victorian children. Med J Aust 1980; 193.
Mitchell D G, Aldous K M, Ryan F J. Mass screening for lead poisoning: capillary blood sampling and automated Delves-cup atomic absorption analysis. N Y State J Mod 1974; 74:1599-603.
Mitchell D G, Aldous K M, Ryan F J. Mass screening for lead poisoning: capillary blood sampling and automated Delves-cup atomic absorption analysis. N Y State J Med 1974; 74:1599-603.
Rosen J F. The microdetermination of blood lead in children by flameless atomic absorption: the carbon rod atomizer. J Lab Clin Med 1972; 80:567-76.
Parsons, P J, Reilly, A A, and Esernio-Jenssen, D, Screening children exposed to lead: an assessment of the capillary blood lead fingerstick test; Clin. Chem. 42:2 302-311 (1997).
Lyngbye, Jorgensen, Grandjean and Hansen in “Validity and interpretation of blood lead levels: a study of Danish school children”, Scand J Clin Lab Invest 1990; 50: 441-449
   In spite of the various authors' enthusiasm for these approaches, none have been adopted by the CDC, or any State Health Department. While these researchers all produced good results in their carefully controlled studies, there are problems that these methods do not address and some of these methods are not reasonable or feasible to utilize in practice.

A. Soap and Water

Soaps vary widely in their ability to remove the metal contaminants of interest, with removal efficiencies for lead compounds from the upper skin surface ranging from 5% to 50% for commonly used soaps. The formulators of soap and liquid skin cleaners do not design their products to be efficient at removing metals to the low levels required for sampling of the blood through the skin for measurement of the metal concentration.

Soap is the product of the reaction between a fatty acid or a fatty acid ester and an alkali. This is known as the saponification reaction. Natural soaps are produced by the reaction of animal or vegetable fats and alkali. Synthetic soaps are produced from other fatty acids and alkali. A detergent is any substance that breaks and reduces the surface tension of water, i.e. makes the water ‘wetter’. All soaps are detergents. Not all detergents are soaps. Detergents are typically composed of one or more surfactants (surface active agents). Soaps and detergents also emulsify (break and disperse in water) oils and greases. Surfactants and soaps both have one end of the molecule attracted to water (hydrophile) and the other end is a long non-polar hydrocarbon chain that is attracted to oil, and grease (hydrophobe). Surfactants are classified as anionic, cationic, non-ionic or zwitterionic (contains both a cation and anion that can dissociate in water) depending on the type of atom or molecule that dissociates when it is mixed into water.

In this CDC terminology, it is presumed that the term “soap” is used to broadly include the natural soaps and bar soaps as well as liquid skin cleaners.

Liquid skin cleaners are composed of a mixture of surfactants, detergents and other ingredients. These other ingredients often include a small level of a chelating agent along with small amounts of preservative, moisturizers, colorant, fragrance, and in some products an antibacterial agent.

Soil can be categorized into three broad groups: organic, inorganic and combination. Organic soils encompass a broad range and include food materials, such as fat, grease, protein, carbohydrates, living matter, such as mold, yeast and bacteria and petroleum soils such as motor oil, bearing grease and cutting oils. Inorganic soils include rust, scale, hard water deposits and minerals such as sand, silt and clay. Combination soils contain both organic and inorganic materials mixed together.

Skin cleaners and soaps are designed for the mass markets. The hundreds of surface active agents, detergents and soaps available to the formulator have been studied for many years and their abilities to remove soils, organic, inorganic and combination is well known. Producers of skin cleaners are concerned with the following requirements when they create a skin cleaner. The typical product objectives are:
1. Skin to be visibly clean with commonly encountered skin contaminants.
2. Ease of rinsing, or ‘free rinsing’.
3. No or low residue—i.e. no ‘latent residue’.
4. Non-irritating to the skin.
5. Esthetics—color, fragrance, viscosity, clarity and lather.
6. Cost—is the most important consideration today in the design and production of skin cleaners.

Some skin cleaners are designed for specific purposes, e.g. antibacterial skin cleaners. Antibacterial skin cleaners are required to meet minimum bacteria kill rates.

Formulators are concerned with meeting the visibly clean standard, free rinsing, non-irritating and esthetic standards with the use of their products at the lowest cost. The skin is frequently covered with dirt, grease, cooking oils, fats and sebaceous gland oils which can be tens to hundreds of microns thick. These are the contaminants common soaps and skin cleansers are designed to remove. Less than 10% of the US population is exposed to lead at a potential level that would result in a blood lead level of concern. Efficient removal of the smallest traces (nanograms) of lead and other metals does not enter into the evaluation of the visibly clean standard. Lead oxide on the skin for example is completely invisible to the naked eye at levels of 1 microgram per mm² (1,000 nanograms per mm²).

All of the published capillary blood sample protocols specify the stick site is to be washed with soap and water. Soap is the water-soluble reaction product of a fatty acid and an alkali. Soap is actually a specific type of salt, where the hydrogen of the fatty acid is replaced by a metal, typically sodium. Soap lowers the surface tension of water and permits the emulsification of fat-bearing soil particles. Soaps are particularly poor at removing most of the metal contaminants of interest in blood samples from the skin. Soaps are only marginally effective at wetting many metals and particularly metal oxides, and they form precipitates with many metal ions depositing them onto the surface. This is commonly observed as soap scum. When the action of soap on a thick layer of metal or metal oxide dust on the skin is observed closely, one sees the soap form a layer over the top of metals on the skin, and smears over the top of them, without penetration or lifting, two steps required to remove any soil off a surface. Common soaps are also poor at exfoliation of dead skin cells. Exfoliants are a separate and special class of skin cleaners that do not exhibit the surface active, surface tension and wetting properties of soaps and skin cleansers.

Many skin cleaners and soaps contain small amounts of a chelating agent, most typically a sodium salt of ethylenediaminetetraacetic acid (EDTA). They function as water softeners to remove the water hardness ions of calcium, magnesium, iron and manganese. These ions interfere with the cleaning ability of soaps and surfactants and act like dirt and “use up” and precipitate the surfactants, using up an excessive portion of them, making them unavailable to do the soil removal job desired. Chelating agents are less expensive than the equivalent amount of surfactants that would be required to remove the water hardness, and do not precipitate than onto the surface being cleaned. Chelating agents surround and dissolve the water hardening metal ions in the solution and isolate them so they do not use up the soap or surfactants forming soap scum. This chelating process is very effective, but is not always necessary in skin cleaner formulations intended for most typical purposes and adds to the cost of the formulation. At the normally encountered levels of water hardness, the small amount of added EDTA is more economical than the equivalent amount of surfactant or detergent and often results in a visibly cleaner surface.

Commonly used chelating agents in skin cleaning preparations include in addition to EDTA, citric acid and its salts, sorbic acid and its salts, zeolites, carboxylic acids and their salts and phosphates. Other commercially available chelating agents include Nitriloacetic acid and salts (NTA) [Nitrilotriacetic acid and its salts are possibly carcinogenic in humans (Group 2B)], Hydroxyethylenediaminetriacetic acid and salts (HEEDTA), Diethylenetriaminepentaacetic acid and salts (DTPA) and Diethanolglycine and salts (DEG), Ethanoldiglycine and salts (EDG), Hydroxycarboxylic Acids and salts (HCA), such as Citric Acid and its salts, Gluconic Acid and its salts, Ethylenediamine (EDA), Diethylenetriamine (DETA) and Aminoethylethanolamine (AEEA) and ethyleneamines. In addition, acetic acid and their salts also act as chelants under certain conditions. This group of chelants is not normally used in skin cleaning preparations. The phosphates are sometimes used in non-skin cleaning applications, e.g. laundry detergents.

According to the brochure of a major producer of chelating agents, the benefits of incorporating a chelating agent into skin cleaners or soaps include:
1. better lathering in shampoos and soaps, particularly in the presence of hard water,
2. improved shelf life
3. preventing softening, brown spotting and cracking in bar soaps
4. improved stability of fragrances, fats, oils and other water soluble ingredients.

Builders are added to a cleaning formula to upgrade and protect the cleaning efficiency of the surfactants and/or soap; and are a lower cost alternative to cheating agents in the formula. They do a variety of functions including buffering, softening and emulsifying. Builders, in addition to softening, provide a needed level of alkalinity and buffers to maintain the proper pH balance.

Builders soften water by deactivating hardness minerals (the metal ions calcium, magnesium, iron and manganese by chelation, sequestration or precipitation. Both chelation and sequestration hold metal ions in solution.

Chelation occurs when the chelating molecule captures the metal ion and incorporates it inside the molecular structure. Sequestration is similar, but in this instance, when it captures the metal ion, it holds the metal ion on the outside of the molecule. Precipitation is removing these ions from solution as insoluble materials. It should be noted, that the terms chelation and sequestration are often used interchangeably in the literature, but the accurate terminology is used in this application.

In heavy duty cleaning applications, for example, in laundry detergents, phosphates in the form of sodium tripolyphosphate, sodium orthophosphate or trisodium phosphate, as well as disodium carbonate and sodium silicate have been used for this function of softening, buffering, emulsifying oils and greases and dispersing particles. However, these builders are all too harsh to use in a skin cleaner formula.

Preservatives such as DMDM hydantoin, quaternium compounds, or the parabens—methyl, propyl or butyl are added to prevent bacteria from consuming the organic constituents of the skin cleaner. Antibacterial agents such as Triclosan®, quaternary ammonium compounds, alcohol or parachlorometaxylenol (PCMX) are added when it is desirable to kill bacteria that are not washed off the skin during the cleaning process.

Other ingredients include moisturizers and skin conditioners, along with added color and fragrance to make the product distinctive and more esthetically pleasing to the user and occasionally to mask the odor of the cleaning compounds.

Soaps and formulated mixtures of skin cleaners clean the skin by lowering the surface tension of water to allow the surface active agents to wet the dirt. Organic dirt is lifted and dispersed by the hydrophobic end of the molecule, and inorganics are lifted by the hydrophilic end of the molecule. Mixed organics and inorganics are suspended between the opposite ends of two separate molecules.

Soaps and skin cleaning preparations are limited in their ability to remove many metal contamninates from the skin, and they are only marginally effective for the removal of lead and other metals from the skin. Soaps and skin cleaners commonly in use will disperse the metals and metals compounds to the extent they are not sticky by nature or bound to the surface by static charges. Inorganics that are sticky or accumulate and hold a static charge, e.g. lead oxides, iron oxides and cadmium oxide are not readily dispersed by common soaps or skin cleaners. They can remove the metals by dissolution, which is normally limited by the total chelating and sequestering content of the cleaner. The chelating and sequestering content of the cleaner is used first by the hardness ions, and only then only if there is residual chelating or sequestering capacity remaining can they begin to act on the other metals. Lead on the skin, for example, behaves chemically very much like the calcium ions and can precipitate as soap scum from most soaps and skin cleaning formulations.

Another removal mechanism that occurs with soaps and skin cleaners in common use when applied in a metal removal situation is exfoliation of the dead skin cells. The metals on and in the exfoliated cells are removed with dead cells. All soaps and skin cleaners exfoliate to a limited degree. They typically remove only those dead cells that were nearly ready to flake off without any further assistance. Chemical and mechanical (abrasive) exfoliants are a special class of skin preparations, used to perform this special function.

Readily available surfactants, selected for their above average ability to wet metals and oxides, lower the surface tension of water and dissipate the attractive forces binding the metal contaminants to the surface, when combined with elevated levels of chelants and or sequestrants along with surfactants or other ingredients with antistatic properties can be formulated to produce skin cleaners with maximum metal removing capacity and efficiency. It is beneficial that the individual ingredients selected or when blended into a stable mixture also have a strong ability to deflocculate, disperse, extract and float metal particles. Deflocculation is the breaking apart of large particles into smaller particles to allow them to float in water as colloidal sized particles. In order to eliminate all of the clinically significant sources of blood sample contamination arising during skin penetration, the skin cleaner must be capable of removing even the smallest traces of contaminant.

B. Alcohol Wipes

Alcohol wipes are traditionally used in stick site prep for all manner of tests by the medical community. The alcohol wipes perform three functions:—disinfect, clean and they aid in reducing the size of the skin pores. Skin pores expand and contract as part of the body's thermoregulatory function. As the alcohol evaporates, it cools the skin at the site causing the pores to contract slightly. As a cleaner it performs poorly to remove metals, including lead. This fact is clearly demonstrated and shown in the experimental section.

In the experimental section, venous blood lead samples collected with an alcohol wipe cleaning step are compared with venous blood samples collected according to the current invention. We see that the average level of contamination in the venous samples was 2.4 μg/dL at an average blood lead level of 22.9 μg/dL (12.6%). On average, the blood samples collected with the alcohol wipe contained 577 nanograms of lead sample contamination. This quantity of lead, 577 nanograms is equivalent to 51 lead particles, microns in size, or 50,880 lead particles 1 micron in size that contaminated the samples.

C. Barrier Sealants

Barrier films have been tried with and without soap and water after the alcohol wipe. Barrier films or sealants such as silicone or rubber seal the exterior skin surface and are only effective at isolating the blood drop from lead on the topside of the uppermost skin surface while the drop forms. This approach does not address the case where lead is present at the stick site and is pushed by the lancet through the skin into the blood flow. It does not address the presence of lead in, under and between the keratin cells that the blood contacts during its journey to the surface. It does not address the contact of the blood sample with the potentially contaminated walls of the wound or the lead in the skin fragments that are scraped off the sides of the wound, or the extracellular and intracellular fluids incorporated into the blood sample. It adds another step to the collection process.

D. Acid Wash

Washing of the skin with dilute acetic acid (vinegar) or dilute nitric acid has been tried as an option following the soap and water wash. Both of these acids dissolve lead and most of the trace metals of interest to a high degree. A nitric acid wash is very effective for non-porous surfaces that have already been washed with detergent. It is frequently employed in the laboratory to assure that glassware, sampling supplies and collection containers are free of trace metals. However, in this procedure for cleaning laboratory supplies, the nitric acid wash is typically followed by a triple rinse with distilled or de-ionized water.

These acids are polar (charged) molecules and do not have the ability to wet skin or skin oils (non-polar molecules) or to penetrate into the stratum corneum layer and dissolve the metal located below the surface. Some acids, such as nitric can oxidize or destroy skin oils. Acid can only address the metal contamination on the 2-dimensional outer surface of the skin. No penetration occurs unless sufficient concentration and time are used to corrode the upper skin layer. As discussed previously water soluble metal salts, such as lead acetate and lead nitrate diffuse very rapidly through the sweat ducts and somewhat slower through the stratum corneum. A significant portion of the lead dissolved by the acids will subsequently contaminate the skin layer where it can come into contact with and contaminate the blood sample. In addition, the removal of these salts onto a cotton or paper wiping substrate is not effective, as there is no method for binding water soluble metal salts to the fabric and preventing them from being smeared across the surface. One researcher used 0.3 N nitric acid (18.9% HNO₃ by weight). Checking multiple sources for handling nitric acid safely all state: “Do not allow even dilute nitric acid solutions to come into contact with your skin.” The acid washing procedure also adds another step to the sample collection.

E. Special Blood Sampling Devices

Additionally, certain specialized sampling devices have been developed that attempt to reduce the contamination of a blood sample. One particular device includes a separate catheter positioned around a needle. After venipuncture, the needle can be withdrawn from the catheter such that blood collection occurs through the catheter to avoid contact with skin and metals. However, as discussed previously, this device does not address the case where lead is present at the stick site and is pushed by the needle and/or catheter through the skin into the blood flow. It does not address the presence of lead in, under and between the keratin cells incorporated into the sample. It does not address the contact of the blood sample with the potentially contaminated walls of the wound or the lead in the skin fragments that are scraped off the sides of the wound into the blood sample or the extracellular and intercellular fluids incorporated into the blood sample. It adds cost and increases complexity of the sample collection.

The need for preventing contamination during sample collection has long been recognized, but all previous work has approached the problem of preventing sample contamination as a 2 dimensional surface problem of removing lead from external environmental sources. The present invention addresses both an improved methodology for removing sources of contamination on the outer skin surface, as well as sources of contamination that exist within the skin layer that have not previously been taken into account.

In summary, current sample site preparation protocols for venous blood samples address only disinfection and do not remove the contaminants that increase the analytical result above the true value. Current sample site preparation protocols for capillary blood samples address disinfection and general cleanliness, but they do not effectively address removal of the surface and subsurface contaminants. This is because existing stick site cleansing protocols do not effectively deal with surface contamination on the outermost layer of the skin and friction ridges, and ignore the need for deep cleaning of the pores, the porous desiccated skin cells, the sweat glands and hair follicles. These structures and surfaces frequently contain levels of the metal(s) to be analyzed. As a result, the capillary protocols incorrectly presume the use of soap and water is an effective means to remove metal contaminants from the skin surface. Current blood sample protocols for both capillary and venous samples solely address sources of contamination on or above the skin surface, i.e. the 2-dimensional outer surface, and ignore the presence of subsurface, i.e., 3-dimensional, contamination and do not provide an adequate means of reducing and controlling blood contamination from these 3-dimensional sources.

Therefore it is desirable to develop a simple, fast and improved method for the removal of metal contamination from the exterior skin surface as well as the removal of subsurface contamination that falsely raises the measured concentration of metals in blood samples. The desired method would involve the cleansing of the stick site with a skin cleanser and/or skin cleansing wipe (hereinafter “anti-static metal sequestering skin cleaners”) that is highly effective at wetting, releasing, sequestering, complexing, extracting, breaking static attractions, dispersing, deflocculating and floating the metal contaminants from the surface of the skin as well as penetrating, extracting and acting upon the contaminants located on the interior surfaces of the skin pores and structures that are open to the surface. This is an improved and a highly effective method for reducing blood sample contamination and improving the accuracy of measurement of the metals content of blood samples. To assist in the removal of the contaminants from the skin, it is further desirable that the method involve the exfoliation of the skin surface to some degree.

In addition, two of the difficulties in obtaining a capillary blood sample are inadequate blood flow and premature clotting. The formation of blood clots requires available calcium ions. Therefore, it is also desirable to develop a method capable of reducing the calcium ion concentration on the skin surface and in the skin surface to aid in reducing the speed at which the blood clots for several seconds and to assist the practitioner in obtaining the needed blood volume.

### SUMMARY OF THE INVENTION

According to a primary aspect of the present invention, a method for obtaining a blood sample is provided in which, prior to collecting the venous, capillary or arterial blood sample for analysis, the skin is first cleaned with one or more specially formulated liquid, gel or solid type skin cleaner(s) with a demonstrated high removal capacity for the chemical species to be analyzed in the sample. Typically skin cleaners of these types will be specifically designed and formulated for maximum removal capacity, efficiency and efficacy for the species of interest. In the case of metals analysis of blood samples, one or more such formulated skin cleaners are used singly or in sequence, to reduce both surface and subsurface metal contamination, i.e., reducing the potential contamination of the blood sample as it is drawn through the 3-dimensional section of the skin. More particularly, the cleaning method and materials of the present invention remove metal contaminants from the surface of the skin, as well as drawing subsurface contaminants out of the skin pores, sweat ducts, sebaceous glands, hair follicles and the intercellular spaces between the skin cells for subsequent removal from the skin surface. The method and materials of the present invention also remove the metals located within, on and between the desiccated epidermal cells, along with removing multiple layers of the dead epidermal cells by exfoliation of the contaminant-containing desiccated epidermal cells from the upper surface of the skin. This method significantly reduces the error rate in the measured level of lead in blood, resulting in a more accurate measure of the blood lead concentration for both capillary and venous samples. The method can also be used for improving the accuracy of measurements for all of the metals of interest in blood, including, but not limited to: cadmium, iron, cobalt, calcium, copper, mercury and potentially as analytical methods improve arsenic content of the blood. This is not an exhaustive list, but these metals are listed by way of example. This method can be extended to include potassium, when the cleaners are formulated with only sodium or ammonium as the cation in the cleaning formulas; and can be extended to include sodium, if the cleaning compounds are formulated with ammonium or more expensive potassium in place of the sodium in the formulation. In the special case of these and other highly water soluble contaminants, it is more effective to use do-ionized or distilled water for water rinsing steps than tap water since distilled and de-ionized water do not contain any metal ions. Improved removal of the contaminant(s) to be subsequently analyzed from all of these skin surfaces, interior and exterior, surface and subsurface, provides a sample that produces an analytical result that more closely reflects the true concentration of the metal of interest in the blood and improves the accuracy and precision of the measurement. The blood sample contacts a surface area that is thousands of times larger than the 2 dimensional surface that is penetrated. Consideration of the subsurface structures in the skin layer adds a third dimension with a large surface area where the contaminant(s) to be measured frequently reside. It is necessary to clean all of these skin surfaces to the maximum extent possible to reduce contamination of the blood sample. With this improved method, and additional research, sufficient accuracy may be achievable with less invasive (fingerstick) blood specimen collection to extend the use of capillary blood samples into areas that currently only utilize venous samples e.g. occupationally exposed individuals.

According to another aspect of the present invention, these specific surface and subsurface cleaning steps of the method of the present invention may occur simultaneously or sequentially during the cleaning process. The main steps of the method and materials used therein which can occur simultaneously or separately in various methods, are:
1. Removal of any heavy surface loading of the contaminants by wetting, static charge dissipation, breaking the surface adhesion forces, sequestering and/or chelating, followed by deflocculating, dispersing and floatation followed by rinsing and/or adsorption and absorption into and onto a substrate (towel or cloth for example).
2. Exfoliating multiple layers of the dead desiccated outer skin cells holding these contaminants within their porous structures. Steps 1 and 2 also open blockages that often exist at the surface openings of the pores, sweat ducts and hair follicles allowing the next steps to be more efficient and effective.
3. Drawing the subsurface contaminants out of the pores, sweat ducts, hair follicles and out of the remaining dead desiccated cells up and onto the surface by penetration, wetting, static charge dissipation, breaking of the adhesion, sequestering or chelating, deflocculating, dispersing, extraction and floatation.
4. Removal of these raised subsurface contaminants from the outer surface after they have been detached and drawn out of the subsurface structures up and onto the outer surface by wetting, static charge dissipation, breaking the surface adhesion, sequestering or chelating, deflocculating, dispersing and floatation followed by rinsing and/or adsorption and absorption into and onto a substrate.

According to still another aspect of the present invention, the method and materials include cleansing the stick site with a formulated cleaning solution applied to a fabric substrate that can draw additional metal contamination out of the skin pores, sweat ducts, and hair follicles, even below the skin surface and remove a further portion of the subsurface metals after they that have been raised to the surface. Additionally, the fabric substrate in conjunction with the impregnated cleaning solution should be capable of binding the metals to the fabric so that they do not smear or spread the contaminants on the surface. The substrate is selected to provide gentle mechanical abrasion to remove or exfoliate additional layers of dead skin cells.

According to a further aspect of the present invention, as a final skin penetration preparation step in the method, the stick site can be disinfected with an alcohol wipe. This additional step can be skipped if the previous steps incorporate a demonstrated disinfection capability. However, it appears that the alcohol wipe can provide additional exfoliation of one more layer of the dead cells, as well as reducing the diameter of the skin pores by localized cooling.

According to still a further aspect of the present invention, the method and materials of the present invention are highly efficacious in the removal of calcium ions from the surface and subsurface of the skin. Since blood clotting cannot occur without the presence of calcium, the reduction of the calcium level at a capillary penetration site improves blood flow briefly, making it easier to collect the sample without premature clotting or coagulation. While this has not been studied experimentally at this point in time, observations of hundreds of capillary blood lead samples illustrates that the onset of clotting after this skin preparation procedure is delayed by 5 to 15 seconds, making it easier to collect the sample. This may provide a significant benefit for diabetics, for example, who have to take several capillary blood samples each day to measure their blood glucose level. The particular group that is most likely to benefit from this will be those individuals, regardless of the test, who have difficulty forming a complete drop of blood before coagulation or clotting commences.

Numerous other aspects, features and advantages of the present invention will be made apparent from the following detailed description.

### BRIEF DESCRIPTION OF THE DRAWINGS

In the drawing figures:

FIG. 1 is a cross-sectional view of the layers in the skin on an individual; and

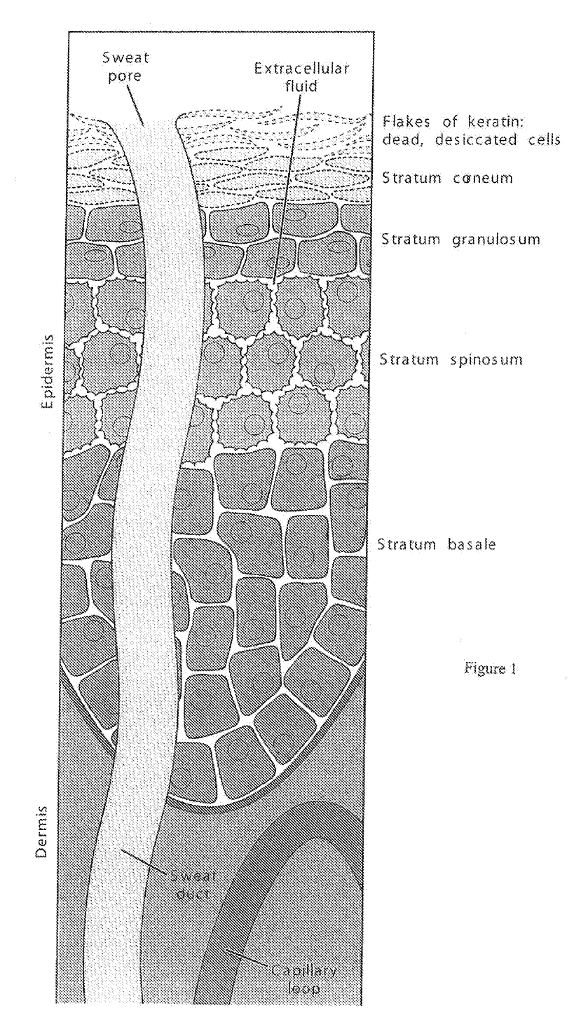

FIG. 2 is a schematic view of a venipuncture being performed through the skin surface of an individual.

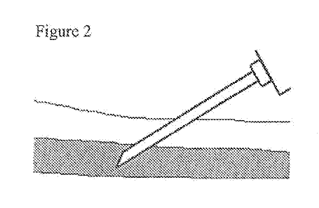

### DETAILED DESCRIPTION OF THE INVENTION

The present invention is a method for the use of certain skin cleaning preparations that are highly effective in the removal of both surface and subsurface contaminants on the skin of an individual in order to enable a blood sample to be obtained from the individual with little or no contamination from contaminants on or in the section of skin through which the blood sample is obtained. The skin cleaning preparations usable in the method are formed at least with: a) a surfactant or a soap; and b) a chelating agent, among other suitable components.

### I. Skin Cleaner Components

A. Surfactants

Surface active agents or surfactants and ordinary soaps are used in cleaning formulations for their ability to: lower the surface tension of the water, wet contaminants and break the adhesive forces.

Some examples of ingredients that when combined in the proper amounts will perform these functions in an efficient manner to best clean and prepare a blood sample stick site include, by way of example:

Surfactants that are efficient providing the properties of good wetting agents for metals, metal oxides and metal salts, and do not form precipitates with the metals commonly tested in blood, include the arylalkyl sulfonates for example the sodium linear alkyl sulfonates. These are classified as anionic surfactants. Sodium dodecylbenzene sulfonate is a particularly good example of an efficient wetting agent with a very good ability to lower the surface tension of water and does not form metallic precipitates. They are particularly good in the formulation of the cleaning compounds described here due these abilities as well as their ability to increase the solubility of other surfactants in the presence of metals, metal oxides and metal salts. Another efficient class of surfactants beneficial to meeting these objectives is the alkyl sulfates of which sodium laureth sulfate and sodium lauryl sulfate are representative members and the alkyl ether sulfates, of which sodium lauryl ether sulfate is a representative member.

Surfactants that are efficient providing antistatic properties include all of the quaternary surfactants. Quaternaries contain at least one nitrogen atom linked covalently to four aryl or alkyl groups. This results in the formation of a positively charged nitrogen atom which is retained regardless of the pH. The types of compounds that provide this form of antistatic property include the Alkylbenzyldimethylammonium salts, such as Benzalkonium chloride, Benzethonium chloride, Steralkonium chloride and Quaternium-63; the betaines, such as the alkyl betaines, alkylamidopropyl betaines and alkylimidopropyl betaines; the heterocyclic ammonium salts, such as Alkylethyl morpholinium ethosulfate and Cetylpyridinium chloride; the tetraalkylammonium salts, the hydroxyalkyl trialkylammonium salts and tetraalkylammonium salts. All of these listed quaternary compounds are cationic surfactants. Another class of antistatic surfactants includes the phosphoric acid esters and salts, for example the anionic surfactant Lecithin and the mono- and d-phosphates which are zwitterionic surfactants. Another potential benefit derived from incorporating quaternary ammonium compounds, such as benzalkonium chloride is its demonstrated antibacterial ability and functionality.

Another surfactant type with excellent antistatic properties is of the non-ionic type known as Amine oxides. The amine oxides in addition to providing additional antistatic performance also are effective at dispersing calcium oxides, magnesium oxides and the other metal oxides with the tendency (like calcium and lead) to produce precipitates with other surfactants and at reducing the skin irritating characteristics of the surfactants listed above that may be used in formulations of this type. Examples of Amine oxides that can provide these functions include Oleyl dimethylamine oxide, Cocamidopropyl dimethylamine oxide, Lauramine oxide, Cocamidopropylamine oxide and Lauryl dimethylamine oxide.

The combination of one or more members of the types of cationic surfactants combined with one or members of the anionic and/or zwitterionic quaternary compounds and/or one or more members of the non-ionic amine oxides provides excellent wetting, lowered surface tension and ability to reduce the static and other adhesive forces that bind metals, metal oxides and metal salts to the skin. When these ingredients are combined with an amine oxide the resulting base skin cleaner formulation is mild to the skin and contains cationic, anionic and non-ionic surfactants in a stable blend with excellent wetting, surface activity, adhesion and antistatic reduction for a wide range of metals and metal compounds as well as good cleaning ability for the broad spectrum of possible or likely skin contaminants.

B. Chelating Agents

1. EDTA

In order to maximize the efficiency of this combination of surfactants, any water hardness present must be controlled. Traditionally this is accomplished by adding a small level of chelating agent Compounds which are commonly used and effective in performing the functions of chelating and sequestering the water hardness metals as well as the other metals of interest in blood samples include by way of example Tetrasodium EDTA and Disodium EDTA, citric acid and sodium citrate as well as the other chelates listed below. Chemical sequesterants, such as the phosphonates which are not often used in skin cleaner formulations also perform this function very well, and in the current instance of concern with maximum removal of the heavy metal, toxic metal, beneficial trace metal and transition metal contaminants actually perform better than the traditional chelating agents due to some of their other unique properties.

Liquid skin cleaners used in the present invention include an elevated level of a chelating agent, such as EDTA or a citrate, and/or an elevated level of sequesterants, such as a phosphonate, in order to perform the function of metal removal from the skin to a better extent than prior art soap or skin cleansers that have 0.05% to 0.25% by weight levels of EDTA. These levels are typically just enough to control water hardness, improve lather, stability and shelf life. Since they are typically completely consumed by the water hardness, there is little or none left to deal with the other metals present. Many of the metals of concern act like water hardness in soap-detergent systems. Chelates, such as EDTA, or the tetra sodium or disodium salts thereof are all effective to a degree. They are available from Dow Chemical Co., for example, under the trademark Versene®. EDTA is a common ingredient of hair shampoos and some skin cleansers. It is added typically to help soften the water by chelating calcium and magnesium atoms. Other chelating agents, including by way of example, NTA, HEEDTA, DTPA, DEG and EDG, but certainly including other chelating agents, can be expected to also provide enhanced metal removal from the skin. However, the level required to accomplish lead removal in the method of the present invention is significantly higher than the typical level found in these common skin cleaners.

However, while EDTA and similar chelating agents can be utilized in the cleaners used in the method of the present invention, the use of EDTA in skin cleaners presents some problems. These include:
1. EDTA appears on the EPA Hazardous Substances List under both the Clean Air Act and Clean Water Act categories.
2. EDTA appears on the California Hazardous Substances List.
3. EDTA is a skin and eye irritant, particularly at the elevated levels necessary to accomplish the desired level of metal removal. (However, this could be overcome by the addition of other ingredients to counter the chelates' irritation properties.)

EDTA is very costly to remove in wastewater treatment, because in order to precipitate any metals in the waste water, the EDTA must be destroyed. However, this is cannot be done on a consistent, economical basis. Chelates delivered to the waste water treatment plant pass right through the treatment process, resulting in the metals being discharged to the receiving waters.

2. Phosphonates

It has been known for some time that cleaners of the types listed herein were effective at removing surface metals contamination along with the full spectrum of dirt and organic and inorganic soils encountered from the outer skin surface. With this method, utilizing the improved formulations of the types listed, in addition to removing surface contaminants, cleaners of this type also are capable of removing sub-surface skin contaminants by penetration and extraction. The use of phosphonates, particularly the organophosphonates, and other sequesterants, for example sorbic acid and its salts, as well as some of the unique properties of some quaternary ammonium compounds, such as benzalkonium chloride, when blended in a stable and compatible manner into quality skin cleaning formulations along with other typical components results in the improved removal of significant amounts of sub-surface heavy metals from the skin pores, sweat ducts and hair follicles.

Phosphonates according to the sales literature of the producers of phosphonates, are “versatile metal ion control agents” with potential uses in any application requiring a hydrolytically stable, water soluble product for sequestering calcium, magnesium and many other metal ions. They form stable molecules with sequestered metals over a broad range of pH. Phosphonates have been and are used in detergents, cosmetics and personal care products. They are used to control hardness ions, such as calcium, magnesium and iron and are very effective dispersants for solid materials to keep them suspended in water.

Phosphonates are more effective at deflocculation, dispersion and anti-redeposition of solids than the other chelating agents commercially available without the skin irritation that accompanies their use. They are as effective as the strongly irritating sodium tripolyphosphates and tetrasodium pyrophosphates at dispersing solid materials into suspensions in water. They also appear to provide an additional means to extract subsurface metals that the traditional chelating agents lack by providing a strong anionic (negative) charge that provides a very strong attraction for positively charged metal ions.

Phosphonates include the acids and salts of Aminotri (methylenephosphonic acid) (ATMP). The CAS name for ATMP is Phosphonic acid, nitrilotris (methylene) tri. Other phosphonates include: 1-Hydroxyethylidene-1, 1-diphosphonic acid (HEDP); Ethylenediaminetetra (methylenephophonic Acid) (EDTMP); Hexamethylenediaminetetra (methylenephophonic Acid), (HMDTMP); and Diethylenetriaminepenta (methylenephophonic Acid), (DETPMP), by way of examples.

Skin cleaners incorporating the types of ingredients listed above are Anti Static Metal Sequestering Skin Cleaners collectively referred to in this disclosure as “Type A” Anti Static Metal Sequestering Skin Cleaners. These Type A Skin Cleaners are water rinse able formulations.

3. Terpenes

Another class of skin cleaners that are effective at performing these functions utilize terpenes, which are essential oils naturally produced by a wide variety of plants. Terpenes have bare oxygen atoms at one end of the long molecule which can acquire and hold a negative charge. This negative charge provides a strong means to attract, lift and hold metals and metal compounds and then hold them in suspension. Formulas utilizing terpenes may be blended with alkyl polyglucoside surfactants (non-ionic surfactants), or with the types of surfactants listed in the formulations of Type A, and together provide the necessary functions of lowering the surface tension of the water, wetting the metal and other contaminants and breaking the adhesive forces binding the metals to the skin surface and subsurfaces. When this base blend is combined with an alkanolamine, such as triethanolamine, an amine oxide and phosphonates, the resulting skin cleaner provides the same or better metal removing capacity as the Type A skin cleaning formulas listed above. The alkanolamine provides the benefits of metal sequestering, anti-redeposition and convert oils present on the skin into soaps.

Skin cleaners incorporating the types of ingredients listed above are Anti Static Metal Sequestering Skin Cleaners collectively referred to in this disclosure as “Type B” Skin Cleaners.

C. Optional Components

Skin cleaners of type A and Type B can also incorporate an abrasive to increase their exfoliation capability. Other components that can be added to these cleaning preparations include a preservative to extend the product's shelf life, moisturizers, humectants or emollients to make the product milder to the skin, colorant and fragrance to make the product more esthetically pleasing and an antibacterial agent to kill bacteria that reside on and in the upper layers of the skin.

### II. Skin Cleaner Formulations

Phosphonate levels, chelate levels and combined levels of chelates and phosphonates that are effective in formulations of type A and B to maximize the metal removal capacity from the surface and subsurface range from 0.25% to 10.0%. They can be effective at levels as low as 0.1% when, for the purposes of this procedure if it is used for very low metal concentration levels and or in conjunction with soft water, do-ionized or distilled water. Formulations of types A and B are effective at meeting the objectives of this invention at levels up to 25%, with very hard water and very high levels of metals present They can be formulated over the entire pH range between 3.5 and 10.5.

While the use of phosphonates is preferred in these formula types, it can be readily understood by practitioners knowledgeable in the field that other chelates used singly or in common have the ability perform some or all of the necessary functions.

A. Examples of Preferred Skin Cleaner Formulations

To achieve a comparable low residual level effectiveness and removal of elevated levels of metals and metal compounds from the surface and subsurface, with EDTA or the other strong chelants (NTA, HEEDTA, DTPA, DEG and EDG); required concentrations appear to range from 0.5% to 25% by weight in the skin cleaning preparations, with 0.5% to 0.75% appearing to be the maximum concentration that avoids the skin irritation that accompanies the use of these chelant types in skin cleaners at levels above about 0.4%. A variety of liquid skin cleansers, commercially available from ESCA Tech, Inc. under the trademark D-Lead® are produced to remove lead, other heavy metals, the transition metals and arsenic from the skin quickly and efficiently, without any EDTA, and have a higher lead and metal removal capacity than other types of skin cleaners. It has unexpectedly been discovered that these formulations, as well as formulations of similar types are effective at removing not only surface skin contamination, but also the sub-surface skin contaminants of concern in the methods for collection of both capillary and venous blood samples.

These products include D-Lead® Hand Soap, item #: 4222ES, D-Lead® Deluxe Whole Body Wash and Shampoo, item #: 4224ES, D-Lead® Abrasive Hand Soap, item #: 4229ES and D-Lead Moisturizing Shower Gel, item #: 451ES, (Type A), which all have very high removal capacities for lead, cadmium, mercury, cobalt, nickel, silver, radium, uranium and other heavy metals, as well as calcium and magnesium for example.

All of these products are capable of complete removal of as much as 400 micrograms of lead oxide placed on the hands in a single 20 second wash and 10 second rinse in controlled lab tests. Tests with soaps containing EDTA at elevated levels removed about ½ to ⅔ of this amount (50% to 66% efficient). These results compare very favorably with, for example, Ivory® bar soap at less than 20 micrograms of lead oxide removal (5% efficient) and Dial® Antibacterial Hand Soap which removed less than 120 micrograms (30% efficient) in the same controlled lab tests.

1. Type A Skin Cleansers

The following skin cleaner formulations have similar surfactant systems, and are classified as Type “A” Anti Static Metal Sequestering Skin Cleaners and are water rinsed skin cleansers.

The label of D-Lead Hand Soap, item #: 4222ES, states: REMOVES LEAD, and: ALSO REMOVES NICKEL, CADMIUM, ARSENIC, MERCURY, SILVER, ZINC AND MOST OTHER HEAVY METALS. The ingredients list on the bottle is: Water, Sodium Laureth Sulfate, Sodium Linear Alkyl Sulfonate, Cocamidopropyl Betaine, Sodium Phosphonate, Sodium Chloride, Cocamide DEA, Parachlorometaxylenol, Propylene Glycol, Fragrance, D & C Red #27.

The label of D-Lead Deluxe Whole Body Wash, item #: 4224ES, states: REMOVES LEAD, and: ALSO REMOVES NICKEL, CADMIUM, ARSENIC, MERCURY, SILVER, ZINC AND MOST OTHER HEAVY METALS. The ingredients list on the bottle is: Water, Sodium Laureth Sulfate, Sodium Linear Alkyl Sulfonate, Cocamide DEA, Cocamidopropyl Betaine, Sodium Chloride, DMDM Hydantoin, Sodium Phosphonate, Parachlorometaxylenol, Propylene Glycol, Fragrance, D & C Orange #4.

The label of D-Lead Abrasive Hand Soap, item #: 4229ES, states: REMOVES LEAD, and: ALSO REMOVES NICKEL, CADMIUM, ARSENIC, MERCURY, SILVER ZINC AND MOST OTHER HEAVY METALS. The ingredients list on the bottle is: Water, Abrasive, Magnesium Aluminum Silicate, Sodium Linear Alkyl Sulfonate, Cocamidopropyl Betaine, Sodium Laureth Sulfate, Quaternium 15, Sodium Chloride, Sodium Phosphonate, Coco Diethanolamide, Lauramine Oxide, D & C Orange #4.

The label of D-Lead Moisturizing Shower Gel, item #: 451ES states: REMOVES LEAD, and ALSO REMOVES NICKEL, CADMIUM, ARSENIC, MERCURY, SILVER, ZINC AND MOST OTHER HEAVY METALS. The ingredients list on the bottle is: Water, Sodium Laureth Sulfate, Cocamidopropyl Betaine, Cocamide MEA, PEG-150 Distearate, Potassium Cocoate, Cocamidopropylamine Oxide, Glycerin, Sodium Chloride, DMDM Hydantoin, Fragrance, Sodium Phosphonate, FD&C #5 Yellow, FD&C #1 Blue.

2. Type B Skin Cleansers

The following skin cleaner formulations may be used with or without a water rinse; they have similar surfactant systems, and are labeled for the purposes of this discussion as Type “B” Anti Static Metal Sequestering Skin Cleaners.

D-Lead® Dry or Wet Skin Cleaner, item #: 4460ES, and D-Lead® Dry or Wet Skin Cleaner with Abrasive, item #: 4455ES (Type B) also may be used since they remove lead and other heavy metals and arsenic from the skin quickly and efficiently, without any EDTA, and have a higher lead and metal removal capacity than other skin cleaners. These products are typically applied to dry skin, washed, then removed with a towel (when no water is available) or may be rinsed off with water. Testing indicates that the lead removal capacity from the hands is in excess of 400 micrograms in a single second wash and 20 second wiping as well as in a single wash and 20 second clean water rinse. In field tests with 11 battery workers, we were able to remove as much as 29.5 milligrams of lead from a single hand in one cleaning with D-Lead® Dry or Wet Skin Cleaner (#4460ES), with an average of 4.54 milligrams of lead removed by the dry method. It was noted that the individual with the 29.5 mg of lead removal from the one hand had extremely rough, dry chapped hands, with a tremendous surface area available for lead from the job as well as from body stores via sweat to accumulate.

The label of D-Lead® Dry or Wet Skin Cleaner, item #: 4460ES states: REMOVES LEAD, and: ALSO REMOVES NICKEL, CADMIUM, ARSENIC, MERCURY, SILVER, ZINC AND MOST OTHER HEAVY METALS. The ingredients list on the bottle is: Water, Natural Organic Oil Blend, Alkyl Polyglucoside, Triethanolamine, Lanolin, Carbomer, Amine Oxide, Sodium Phosphonate, Propylene Glycol, PCMX, Fragrance, FD&C Green #3.

The label of D-Lead® Dry or Wet Skin Cleaner with Abrasive, item #: 4455ES states: REMOVES LEAD, and: ALSO REMOVES NICKEL, CADMIUM, ARSENIC, MERCURY, SILVER, ZINC AND MOST OTHER HEAVY METALS. The ingredients list on the bottle is: Water, Natural Organic Oil Blend, Alkyl Polyglucoside, Abrasive, Triethanolamine, Lanolin, Carbomer, Amine Oxide, Sodium Phosphonate, Propylene Glycol, PCMX, Fragrance, FD&C Green #3.

Formulas of these types also have a very high metal removal capacity and break the adhesion of the metals on the surface of the skin and in the pores of the skin and float the lead off the skin efficiently. They also efficiently mobilize large quantities of metal contaminants from both the surface and subsurface of the skin.

B. Methods of Use of Type A and Type B Skin Cleansers

In a first embodiment of the method, the D-Lead® “Type A” Anti Static Metal Sequestering Skin Cleaners are applied to the skin to wash the area that is to be penetrated to obtain the blood sample. The skin may be either pre-wetted or not. The sample area as well as a large area surrounding the stick site is washed thoroughly for 20 to 30 seconds and then the skin is rinsed with clean water and dried with a towel or cloth that is as free of the metal contaminant(s) of concern as is economically and technically feasible. The water may be hard, soft, do-ionized or distilled. The skin may also be dried with a blower, provided the drying air is free of dust, such as the air quality obtained with the use of high efficiency air filters.

In a second embodiment of the method, the D-Lead® “Type B” skin cleaners are applied to the skin to wash the area that is to be penetrated to obtain the blood sample. The skin may be either pro-wetted or not. It appears that applying this cleaner to dry skin provides the greatest quantity of metal contaminant removal. These cleaners are effective at removing metal contamination with or without water. This is particularly useful when samples must be collected at a location without clean water.

In still a third embodiment of the method, the Skin Cleaner of Type B is applied to the dry skin, and spread with clean gauze, paper towel or cloth to cover the sample area as well as a large area surrounding the stick site. Alternately, it may be spread with clean hands or with clean, gloved hands. The skin cleaner is rubbed over and into the skin. In the case of particularly dry or damaged skin, it may be necessary to apply more of the cleaner, as this cleaner can be adsorbed into skin that is very dry. After the cleaner has had 30 seconds to work, the cleaner along with the metal contaminants is removed by wiping with a clean, low metal content fabric, gauze, paper or cloth. It is beneficial if the substrate selected will bind the metals and provide a level of mild mechanical abrasion to assist in the exfoliation of the dead cells. Alternately, the cleaner and the metal contaminants can be rinsed off with clean water then dried as described for cleaners of “Type A.”

In another fourth embodiment of the method, the skin can be cleaned sequentially with a Type A Skin Cleaner followed by a second cleaning with a Type B Skin Cleaner, or alternately, with type B, followed by Type A. In practical terms, it appears that the major benefits can be obtained by use of either a Type A Skin Cleaner, or a Type B Skin Cleaner, followed by a cleaning with the pre-moistened towel described below.

C. Skin Cleaning Wines

For maximum metal contaminant removal, this washing step around the area of the stick site should be followed with a cleansing with a premoistened wipe of the type described below prior to the alcohol wipe. Premoistened wipes with high lead and heavy metal removal capacity are also commercially available from ESCA Tech, Inc., under the trademark D-Wipe®. The label on the container of D-Wipe® Towels states: —Removes Lead, Nickel, Cadmium, Arsenic, Silver, Mercury, Zinc and most heavy metals form skin and surfaces. It also states: D-Wipe® Towels were specially designed for immediate clean up of lead and metals without water.—Gentle to our skin. The ingredient list states: Deionized water, SD Alcohol 40, Benzalkonium Chloride, Sodium EDTA, Sorbic Acid, Cocamide DEA, Fragrance, Aloe. These wipes do contain Sodium EDTA, which aids in the transfer of metals from the surface and to some extent the subsurface to the fabric, and assist in binding it tightly to the fabric substrate.

In this formulation for the wipes, the ingredients collectively provide the same steps as the Type A and Type B skin cleaners described previously and fulfill many of the same functions, along with some additional benefits. In particular, the same or better performance can be achieved with a combination of EDTA, phosphonates, sorbates and citrates or phosphonates without EDTA, and with or without the citrate or sorbate, or many combinations and concentrations of chelating and/or sequestering agents. Other chelating agents can be used, provided they are compatible and safe for use in a skin cleaner formula, such as NTA, HEEDTA, DTPA, DEG, EDG, citrates and gluconates, by way of example, and is intended to provide examples, but not to be an all inclusive list. Many of these appear to have potential to provide the same, similar or better functionality and performance as the phosphonates. Sequesterants including the phosphonates and phosphonic acids previously listed also will provide a means to transfer metals from the skin and bind them to an appropriate substrate. The sorbate is a mobilizing agent for metals that aids in breaking their adhesion to the skin surface, provides some sequestering functionality and also provides an anti-oxidizing function to the formula components.

The use of benzalkonium chloride, and/or a mixture of quaternary ammonium compounds provide an anti-static function to bleed off static charges that attach metals to the skin surface. Other examples of anti-static agents that can be utilized to fulfill this function are the Polydimethylsiloxanes (PDMS), other silicone derivatives, the betaines and amine oxides.

The ethanol contributes benefits in addition to forming part of the carrier for the other ingredients, it also provides emulsification of the oils and grease, a reduction in the tackiness of the skin surface aiding in the release of the metals and aiding in site disinfection. As discussed previously alcohols have a tendency to shrink the size of the pore openings due to the localized surface cooling that occurs as it evaporates. In these types of formulas, this function is delayed throughout the cleaning/wiping process, as the alcohol does not tend to evaporate until the wipe is removed, allowing skin exposure to the air. Meanwhile, the pores remain open for cleaning and subsurface removal. The wipe substrate should preferably be composed of cotton, cellulose, or other absorbent material, preferably a blend of rayon and polyester that is able to bind the metals to the fabric so they are removed from the surface and not spread by smearing across the skin surface during wiping. Wipes of this type are designated as Anti Static Metal Sequestering Wipes, Type 1. They dry by evaporation, and do not require an additional rinsing or drying step.

### III. Experimental

The use of ordinary soaps or skin cleaners to clean the skin prior to the use of the alcohol wipe to remove heavy metals is less effective than the cleaners of the type described here. Our studies show that ordinary soaps remove insignificant amounts of lead from the skin. Ordinary soap is made from animal or vegetable fats and caustic soda (NaOH). For example Ivory® Bar Soap removes less than 5% of the lead oxide applied to the skin in our laboratory tests. In addition, bar soaps can also transfer the small quantity of lead removed to the next user.

We have also determined that common liquid skin cleansers, e.g. liquid Dial® Soap and SoftSoap® also remove very little lead from the skin, i.e., less than 30%. The reason that these soaps have very little lead removal capacity is that they were designed, formulated and optimized to remove common skin contaminates, such as natural skin oils and ordinary soil. They do not have sufficient anti static or metal sequestering capacity to remove large amounts of metals, or small amounts thoroughly. They are ineffective at deep cleaning metals from the subsurface of the skin. Lead and the other metals behave differently. Soaps and skin cleaners of the types listed as examples above have little ability or capacity to wet most metals, metal oxides and metal salts and float them off or surfaces or out of porous structures. Removal of metals from the skin surface and subsurface requires that the cleaning agent efficiently and effectively wet the metal bearing particles and then float them off the surface, out of the subsurface and up into the rinse water or wiping material. It is also beneficial if the cleaner is able to penetrate and extract subsurface metals.

In the presence of large quantities of metals on and in the skin, it is necessary to sequester or chelate the calcium, magnesium, iron and manganese present on and in the skin originating from environmental sources, from sources generated within the body and in the water used for washing and rinsing. Once these hardness ions and particles are “neutralized” by the sequesterants, chelants and/or surfactants, there must be sufficient capacity remaining after removal of the hardness ions to act on the other metals of concern. The phosphonates in the formulas of Type A and Type B provide a number of novel functions when utilized in skin cleaners to provide both high capacity and enhanced removal of metals from the surfaces of the skin. These properties include: dispersion of solid particles away from and out of the skin surface, penetration, extraction, deflocculation and anti-redeposition. They also provide the ability to peptize, or disperse fine particles and form colloidal suspensions. It appears that this attribute also aids in lifting and dispersing the dead, desiccated skin cells that form the outer surface of the skin, and removing the associated metals contained in the interior of these cells. Phosphonates in these formulas also have very high stability constants for calcium, magnesium, lead, manganese, strontium, barium, iron, cobalt, nickel, copper, zinc, thorium and cadmium, among others.

A. Methods of the Use Skin Cleaning Preparations of these Types to the Preparation of a Blood Sample Stick Site

1. Venous Blood Samples

One method of preparing and cleansing the stick site prior to obtaining a venous blood sample may be done as follows:
1. Prior to entering the room or area where the sample will be collected roll up the shirt sleeves and wash both hands and forearms past the elbow with a Type A Anti Static Metal Sequestering Skin Cleaner. Rinse thoroughly with clean water. Dry with a clean, low lint, low metal towel or cloth. Alternately, the hands and arms may be cleaned with a Type B Skin cleaner first.
2. Proceed to the sample collection area or room, where the stick site is washed by the phlebotomist who inserts the following steps into the standard sampling procedure immediately before application of the tourniquet: The phlebotomist puts on clean gloves and applies approximately 7 mL (¼) ounce of a Type B Anti Static Metal Sequestering Skin Cleaner to the area of the vein to be sampled and with a gauze sponge spreads the cleaner over an area approximately 75 mm in diameter centered on the stick site. Use the gauze sponge to work the cleaner into the skin in a circular motion for 5 seconds. Discard this gauze and this pair of gloves. Allow the cleaner to reside on the skin for 20 to seconds and don a new pair of gloves.
3. If too much of the Type B skin cleaner is absorbed due to very dry skin, apply an additional 7 mL and wait an additional 30 seconds. Remove the skin cleaner with a second gauze sponge. Wipe up in a circular motion from the center outwards. Repeat with a second gauze sponge. Discard the sponges. Discard this pair of gloves. Alternately, the area of the stick site can be cleaned with a Type A skin cleaner with subsequent rinsing and drying steps.
4. Don a new pair of gloves and clean the stick site and area extending out from the stick site to clean a total area of 75 mm in diameter with the stick site as the center of the circle using a pre-moistened towel. Use a Type 1 Anti Static Metal Sequestering Wipe Towel. Fold the towel to a size no larger than 75 mm×75 mm and clean from the center outwards in a circular motion. Use gentle pressure to exfoliate dead cells.
5. Discard the wipe and don a new pair of gloves to proceed with the tourniquet and alcohol wipe steps and collecting the blood sample.
6. In an occupational setting, it will be advantageous to have the individual shower and wash their entire body with a Metal Sequestering Skin cleaner of Type A and change into clean clothes in place of washing only the hands and arms.
7. In another variation of this procedure, an Anti Static Metal Sequestering Wipe of Type 1 is used immediately before the Type B skin cleaner to exfoliate dead skin cells and assist in unblocking the pores.
8. The various cleaning formulas disclosed in this application may be used in a different order and this example illustrates some of the many possible variations of the method that will be effective. The practitioner can readily see from this example that different variations of this procedure have the capability of producing the same or similar end results.

2. Capillary Blood Samples

a. One method of preparing and cleansing the stick site prior to obtaining a capillary blood sample may be done as follows:
1. If a finger is to be the sample location—both hands are washed by either the patient or the phlebotomist with a Type A Metal Sequestering Skin Cleaner. Rinse thoroughly with clean water. Dry with a low lint, low metals towel or cloth. If washed by the phlebotomist, then the phlebotomist should don a new pair of gloves first.
2. If the ear lobe, heel or toe is to be the stick site, then the phlebotomist wearing a new pair of gloves washes the foot or the ear with a Type A Metal Sequestering Skin Cleaner. Rinse thoroughly with clean water. Dry with a low lint metals free towel or cloth.
3. The phlebotomist dons a new pair of gloves and cleans the finger, heel, toe or ear lobe around the stick site with a Metal Sequestering Wipe of Type 1. The towel should be folded to a size no larger than 75 mm×75 mm and an area extending beyond the stick site is cleaned with gentle pressure and a circular motion, with the stick site as the center and wiping outwards.

b. Another method for cleaning the stick site is:
1. The phlebotomist wearing a new pair of gloves washes the hand, foot or the ear according to the location of the stick site with a Type B Metal Sequestering Skin Cleaner using between 3 and 7 mL of skin cleaner. Apply the skin cleaner with a new cotton gauze sponge, or other mildly abrasive fabric that is both absorbent and adsorbent, working from the center of the stick site outwards for 5 seconds. Allow the cleaner to work for 20 to 30 seconds.
2. The phlebotomist should don a new pair of gloves and with a new cotton gauze sponge remove the skin cleaner with a circular motion from the center outwards, while applying gentle pressure.
3. The phlebotomist then dons a new pair of gloves and cleans the finger, heel, toe or ear lobe around the stick site with a Metal Sequestering Wipe of Type 1. The towel should be folded to a size no larger than 75 mm×75 mm and an area extending beyond the stick site is cleaned with gentle pressure and a circular motion, with the stick site as the center and wiping outwards.

B. Efficacy of certain Skin Cleaning Compounds at Removal of Lead

Askin, D P and Volkmann, M in “Effect of Personal Hygiene on Blood Lead Levels of Workers at a Lead Processing Facility”, Amer. Ind. Hyg, Assoc. J, (1997), 752-753 used a product that is now commercially available as D-Wipe® Towels to measure the amount of lead on the right hand of workers and found a highly significant correlation between the quantity of lead recovered from the hand and the worker's blood lead level. (Positive correlation coefficient was 0.61 and p<0.002). These tests also demonstrated the ability of the D-Wipe® Towels to remove lead from the hands. One D-Wipe® Towel recovered as much as 4.41 mg of lead from a single hand of a lead worker.

1. Comparison of Lead Removal of Alcohol Prep Pad and D-Wipe® Towel

### Purpose

We have also evaluated the effectiveness of an isopropyl alcohol wipe in removing lead from the surface of the skin and compared it to the effectiveness of the D-Wipe® Towel in removing lead from the skin. Askin, D., Dorko, Z s. and Erdelyi, O. (not published) tested 22 battery workers during a work day.

### Procedure

The amount of lead removed from the inside of the elbow was determined for alcohol prep pads and D-Wipe® Towels for 22 battery plant workers. Workers reported to the cafeteria during their work shift. After cleaning their hands, they were instructed to roll up their shirt sleeve. The technician selected a visible vein inside the elbow and cleaned a 1″×2″ area, centered on the selected stick site, with an alcohol prep pad, simulating the procedure to prep the stick site for a venous sample. The pad was subsequently analyzed for total lead by ICMS.

Then, a D-Wipe® Towel was folded into a 1″ square, and the exact same area was cleaned again. The D-Wipe® Towel was subsequently analyzed for total lead by ICMS.

In 21 of 22 individuals sampled, more lead was recovered with the D-Wipe® Towel than with the alcohol wipe. The amount of recovered lead with the alcohol wipe ranged from 0.5 to 200 micrograms of lead with an average of 23 micrograms. The amount of lead recovered with the subsequent cleaning of the same area with a D-Wipe® Towel ranged from 3.3 to 460 micrograms, with an average of 47 micrograms.

### Results

| | | |
|---|---|---|
| Average lead removed by Alcohol Prep Pad: | 23 | micrograms |
| Average lead removed by subsequent | 47 | micrograms |
| D-Wipe ® Towel: | | |
| Range of Lead removed by Alcohol Prep Pad: | 0.5 to 200 | micrograms |
| Range of Lead removed by subsequent | 3.3 to 460 | micrograms |
| D-Wipe ® Towel: | | |

Results are listed in the order of increasing blood lead level, based on the last test result for the subject.

TABLE 4
Lead Removed by Alcohol wipe and D-Wipe ® Towel

| | Alcohol | D-Wipe ® | | Blood |
|---|---|---|---|---|
| # | Wipe | Towel | Total | Lead |
| Test | μg Lead | μg Lead | μg Lead | Level |
| Subject | Recovered | Recovered | Recovered | μg/dL |
| 2 | 0.5 | 3.3 | 3.8 | 7 |
| 20 | 0.8 | 6.0 | 6.8 | 10 |
| 18 | 1.4 | 9.2 | 10.6 | 11 |
| 16 | 16.0 | 65.0 | 81.0 | 12 |
| 14 | 12.0 | 24.0 | 36.0 | 13 |
| 10 | 0.7 | 4.3 | 5.0 | 14 |
| 7 | 2.4 | 5.2 | 7.6 | 14 |
| 12 | 25.0 | 72.0 | 97.0 | 14 |
| 9 | 25.0 | 40.0 | 65.0 | 15 |
| 15 | 0.9 | 4.8 | 5.7 | 16 |
| 21 | 0.5 | 3.9 | 4.4 | 17 |
| 19 | 0.5 | 4.3 | 4.8 | 18 |
| 8 | 11.0 | 22.0 | 33.0 | 18 |
| 6 | 11.0 | 16.0 | 27.0 | 19 |
| 13 | 15.0 | 64.0 | 79.0 | 19 |
| 17 | 1.4 | 9.2 | 10.6 | 21 |
| 11 | 18.0 | 31.0 | 49.0 | 22 |
| 1 | 29.0 | 46.0 | 75.0 | 22 |
| 5 | 19.0 | 38.0 | 55.0 | 27 |
| 4 | 12.0 | 22.0 | 36.0 | 28 |
| 22 | 200.0 | 460.0 | 660.0 | 28 |
| 3 | 112.0 | 90.0 | 202.0 | 39 |
| Averages: | 23.4 | 47.2 | 70.6 | 18.4 |

### Observations

From the skin of 21 of 22 individuals, the D-Wipe® Towel removed more lead from the skin of the sample area than the alcohol prep pad. It is also interesting to note the tendency of the amount of lead removed from the skin at the stick site to increase with increasing blood lead level. In general, the higher the blood lead level, the higher the amount of lead recovered from the skin. This is a very strong indication of the recovery of subsurface lead which could have originated from the excretion of body stores. It also indicates the quantity of potential sample contamination increases with blood lead level. The higher the individual blood lead level, the higher the potential for more lead to be present at the stick site and the higher the amount of lead that was recovered from the stick site.

For one individual, the alcohol wipe removed more lead than the D-Wipe® Towel. Two observations were recorded on this individual at the time: (1) he had the highest blood lead level in the group: a blood lead level 11 μg/dL higher than anyone else. (2) His skin texture was noticeably different—he had very moist, tight skin with very few ridges or wrinkles, i.e., a much lower total surface area than the other individuals, no hair on his arms and very small skin pores.

### Conclusion

Cleaning the stick site with a D-Wipe® Towel prior to the alcohol prep pad will result in more lead removal from the area of the stick site than the alcohol prep pad normally used. The D-Wipe® Towel has a superior ability to mobilize lead so that it can be absorbed onto and into the wipe substrate, where it can be firmly bound to the fabric. The D-Wipe® Towel must be mobilizing lead that was inaccessible to the alcohol wipe. Based on the subsequent venous sample study, it appears that this process works well with the D-Wipe® Towel wiping followed by the alcohol wipe.

2. Lead Removal Capacity of D-Lead® and D-Wipe® Skin Cleaners

### Purpose

We purchased two dozen commercially available soaps and skin cleaners and twenty commercially available pro-moistened skin cleaner wipe towelettes and compared their lead removal capacity to the removal capacity of D-Lead® Skin Cleaners, Type A and Type B and D-Wipe® Towels. The purchased products were selected to represent a wide variety of formulation types based on the ingredients listed on the product labels. The purchased cleaners were evaluated for their removal capacity for lead oxide from the skin. The purchased skin soaps and cleaners were compared to the D-Lead® formulations and the D-Wipe® Towels were compared to the purchased towelettes.

### Procedure

For the skin cleaner tests, a measured amount of lead oxide (PbO) was applied to the palm of one individual, who then massaged the material into the palm with the opposite index finger. The hands were then rinsed with warm water for 10 seconds, with no attempt to measure the amount that rinsed off with the tap water. Then 4 mL of the liquid soap was applied and the hands washed for 20 seconds, followed by a 10 second rinse.

For the skin cleaners and soaps, the amount of lead remaining on the palm of the closed hand was then tested by applying a chemical spot test (D-Lead® Lead Test Kit, mfg by ESCA Tech, Inc., Milwaukee, Wis.) directly on the palm of the hand. This test turns lead and lead compounds a bright yellow color, and has a visible detection limit of micrograms as Pb. The % removal efficiency was estimated based on a semi-quantitative scale developed by recovering the lead from the first 15 tests with a D-Wipe® Towel and analyzing them for total lead.

### Results

For the purchased skin cleaners, the lead residue remaining ranged from a low of approximately 95% to 50% (removal rate of 5% to 50%). For all of the D-Lead® Skin Cleaners listed as type A and type B, no detectable lead remained on the palm or the opposite forefinger.

For the wipes, the same procedure was used and for the purchased wipes, the lead residue remaining ranged from 97% to 15% (removal rate of 3% to 85%). For the D-Wipe® Towels no detectable lead residue remained on the skin.

3. Field Performance Testing of D-Lead® Skin Cleaners. Types A and B and D-Wipe® Towels

We tested the performance of one Type A and one Type B skin cleaner on 20 battery plant workers. During their work shift, individuals reported to the training room to determine how much lead they had accumulated on their hands while working. D-Wipe® Towels, D-Lead® Deluxe Whole Body Wash (#4224ES) and D-Lead® Dry or Wet Skin Cleaner (#4460ES) were used in the tests.

a. Deluxe Whole Body Wash Group

### Procedure

Nine (9) of the workers were brought into the test room without their gloves directly from the production floor without washing. The left hand of each worker was cleaned three times with successive D-Wipe® Towels by a technician, up to their wrist. After their left hand was cleaned, these workers washed both hands with #4224ES, D-Lead® Deluxe Whole Body Wash and Shampoo. They rinsed their hands for 10 seconds, then 7 mL of soap was applied, they washed for 20 seconds up to their wrists and rinsed for 10 seconds. (The amount of lead removed was not determined, as it was contained in the rinse water). The amount of lead remaining on their right hand was determined with three successive cleanings of their right hand up to their wrist with three separate D-Wipe® Towels. For each individual, the three pre-wash D-Wipe® Towels were combined into one sample container and the three post wash towels were combined into a second container and analyzed by GFAAS for total lead.

### Results

| | |
|---|---|
| Highest level of lead removed with the D-Wipe ® Towels | 3.5 milligrams |
| from the first (left) hand for these 9 workers: | |
| Average amount of lead on the left hand of these 9 | 1.2 milligrams |
| workers: | |
| Average amount of Lead on right hand after washing | 0.04 milligrams |
| with D-Lead ® Deluxe | |

TABLE 5
Lead Removal Capacity of D-Lead ® Deluxe

| | | μg Lead Recovered | |
|---|---|---|---|
| | μg Lead | from right hand after 1 | |
| Test | on Left Hand | wash with D-Lead | % |
| Individual # | before Washing | Deluxe | Removed |
| 1 | 37.4 | ND | 99.9% |
| 2 | 43.7 | ND | 99.9% |
| 3 | 167.3 | ND | 99.9% |
| 4 | 205.5 | ND | 99.9% |
| 5 | 781.5 | ND | 99.9% |
| 6 | 1,174.3 | 124.0 | 89.4% |
| 7 | 1,507.3 | 70.7 | 95.3% |
| 8 | 3,388.8 | 2.8 | 99.9% |
| 9 | 3,492.0 | 130.6 | 96.3% |
| Avg.: | 1,199.8 | | 97.8% |

* Minimum detection limit [MDL] = 20 μg by GFAAS ND = Non Detectable

### Conclusion

With proper washing technique, 97.8% of the estimated lead on their hand was removed with a single hand wash with skin Cleaner Type A, #4224ES, D-Lead Deluxe Whole Body Wash and Shampoo.

b. Dry or Wet Skin Cleaner Group

### Procedure

Ten workers were brought into the test room without their gloves directly from the production floor without washing. After cleaning their left hand with the three D-Wipe® Towels according to the procedure described above, their right hand was cleaned by the technician. The technician dispensed 7 mL of #4460ES, D-Lead® Dry or Wet Skin Cleaner onto their right hand. The cleaner was applied to their dry hands by the technician, and the technician wearing a fresh pair of vinyl gloves for each individual, massaged and cleaned their hand for 20 seconds. Then, the cleaner was removed with a 4″×4″ cotton gaze sponge. The gauze was then analyzed by GFAAS. The right hand was then cleaned again with a D-Wipe® Towel and analyzed for lead. The results of the analysis of the gauze were used to determine the amount of lead removal.

### Results

| | |
|---|---|
| Highest lead recovered from right hand with #4460ES: | 29.5 milligrams |

TABLE 6
Removal Capacity of D-Lead Dry or Wet

| | μg Lead | μg Lead Recovered | % Additional |
|---|---|---|---|
| Test | recovered from left | from right hand after 1 | Lead Removed |
| Individual | hand with D- | wash with D-Lead Dry | by D-Lead |
| #: | Wipe Towels | or Wet | Dry or Wet |
| 10 | 9,419.7 | 29,505.2 | 213.2% |
| 11 | 2,017.5 | 5,534.8 | 174.3% |
| 19 | 1,440.7 | 4,802.3 | 233.3% |
| 13 | 1,651.2 | 3,437.2 | 108.2% |
| 14 | 958.0 | 2,571.5 | 168.4% |
| 15 | 1,970.8 | 2,505.0 | 27.1% |
| 18 | 262.1 | 553.6 | 111.2% |
| 17 | 113.1 | 363.7 | 221.6% |
| 18 | 79.0 | 290.4 | 267.6% |
| 19 | 55.7 | 240.5 | 331.8% |
| 20 | 59.1 | 100.7 | 70.4% |
| Averages: | 1,638.8 | 4,536.8 | 175.2% |

### Observations

The amount of lead recovered from the hand with the Dry or Wet Skin cleaner was highest for those individuals with dry, cracked, rough skin. This corresponded with the net total surface area, that is, the higher the surface area, the higher the amount of lead present and recovered. It could not be determined if the lead removal capacity of the D-Lead® Dry or Wet Skin cleaner is actually superior to the lead removal capacity of the D-Wipe® Towels; or if there was this much difference in the lead loading between the two hands, or if D-Lead Dry or Wet Skin Cleaner is a superior deep cleaning formula for metals.

### Conclusions

D-Lead Dry or Wet Skin Cleaner appears to remove lead from deep in the skin. Substantial quantities of lead are present on the hands of lead workers even when wearing gloves. Lead level on one hand can exceed 10 milligrams, when the surface area is sufficiently large due to rough cracked skin.

4. Lead Suppression Analysis

### Purpose

To assess whether D-Lead® Deluxe Whole Body Wash and Shampoo, #4224ES; D-Lead® Dry or Wet Skin Cleaner #4460ES; or D-Wipe® Towel liquid materially impacts the blood lead result by suppressing the amount of lead available for analysis resulting in a decreased blood lead analytical result. It would be potentially feasible for the residue of a skin cleaner, if incorporated into a blood sample to result in matrix interference during analysis and suppress the quantity of metal detected.

### Procedures

Control specimens (100 μl blood sample, 900 μl matrix modifier) were prepared and analyzed in the customary manner by Graphite Furnace Atomic Absorption Spectroscopy (GFAAS). The known control values were 6.0, 10.0 and 14.0 μg/dL after dilution. Test samples were aggressively prepared by diluting control specimens using a 1:1 ratio (50 μl sample, 50 μl D-Lead® Product and 50 μl D-Wipe® liquid; and 900 μl matrix modifier) and then analyzed in the customary manner.

### Conclusion

When we compare each of the test sample results with its respective control value, the noted difference for each comparison falls within the detection limits of the GFAAS instrument (+/−1 μg/dL). Based upon the results we concluded that utilization of D-Lead Skin Cleaner, D-Lead® Dry or Wet Skin Cleaner and D-Wipe® Towels do not materially impact the blood lead result or cause any matrix interference.

5. Comparison of the D-Lead®-D-Wipe® Stick Site Cleansing Protocol for Venous Blood Lead Samples vs. the Centers for Disease Control Stick Site Cleansing Protocol

### Objective

To compare the level of accuracy achieved with a D-Lead®-D-Wipe® (DLDW-VP) Venous Stick Site Cleansing Protocol for venous blood lead sample collection with the accuracy of the standard CDC recommended Venous Stick Site Preparation Protocol (CDC-VP).

### Procedure

During scheduled blood lead testing at a lead battery manufacturer, 30 volunteers were recruited to provide two (2) venous samples, one from each arm. Workers were tested during their work shift, and were asked not to wash their hands, arms and face (as is customary anytime they leave the plant floor) prior to coming in for their blood lead test. This is consistent with the CDC protocol. Of the 30 volunteers, 29 were able to supply 2 blood samples. The first sample was collected from their right arm vein according to the CDC protocol as published by the web site: Internet Pathology Laboratory for Medical Education (IPLME). http://medlib.med.utah.edu/WebPath/TUTORIAL/PHLEB/PHLEB.html

For the first sample, collected from their right arm, the IPLME protocol for collecting a venous blood lead sample (CDC-VP) quoted below was followed.

Procedure for Vein Selection:
Palpate and trace the path of veins with the index finger. Arteries pulsate, are most elastic, and have a thick wall. Thrombosed veins lack resilience, feel cord-like, and roll easily.
If superficial veins are not readily apparent, you can force blood into the vein by massaging the arm from wrist to elbow, tap the site with index and second finger, apply a warm, damp washcloth to the site for 5 minutes, or lower the extremity over the bedside to allow the veins to fill.

Performance of a Venipuncture:
Approach the patient in a friendly, calm manner. Provide for their comfort as much as possible, and gain the patient's cooperation.
Identify the patient correctly.
Properly fill out appropriate requisition forms, indicating the test(s) ordered.
Verify the patient's condition. Fasting, dietary restrictions, medications, timing, and medical treatment are all of concern and should be noted on the lab requisition.
Position the patient. The patient should either sit in a chair, lie down or sit up in bed. Hyperextend the patient's arm.
Apply the tourniquet 3-4 inches above the selected puncture site. Do not place too tightly or leave on more than 2 minutes.
The patient should make a fist without pumping the hand.
Select the venipuncture site.
Prepare the patient's arm using an alcohol prep. Cleanse in a circular fashion, beginning at the site and working outward. Allow to air dry.
Grasp the patient's arm firmly using your thumb to draw the skin taut and anchor the vein. The needle should form a 15 to 30 degree angle with the surface of the arm. Swiftly insert the needle through the skin and into the lumen of the vein. Avoid trauma and excessive probing.
When the last tube to be drawn is filling, remove the tourniquet.
Remove the needle from the patient's arm using a swift backward motion.
Press down on the gauze once the needle is out of the arm, applying adequate pressure to avoid formation of a hematoma.
Dispose of contaminated materials/supplies in designated containers.
Mix and label all appropriate tubes at the patient bedside.
Deliver specimens promptly to the laboratory.

The blood lead level results for the venous samples collected by the CDC/IPLME protocol are listed in Tables 7 and 8 and labeled CDC-VP for CDC Venous Blood Sample Stick Site Cleansing Protocol.

The second sample was collected from their left arm vein, and the phlebotomist followed the same procedure as listed above, with the following additional steps, immediately before the application of the tourniquet:

a. Three (3) ml of D-Lead® Dry or Wet Skin Cleanser, formula #: 4460-ES was dispensed from a syringe onto the inside of the elbow over the selected vein, centered on the stick site. It was spread with a sterile cotton gauze sponge. It was allowed to sit undisturbed for 30 seconds, while the phlebotomist donned a new pair of gloves and then was wiped off with a new sterile cotton gauze sponge in a spiral, circular motion from the center of the stick site outwards.

b. After the phlebotomist donned a new pair of gloves, a folded D-Wipe® Towel was used to clean the stick site, also in a spiral, circular motion for 5 seconds.

The blood lead sample results for the venous blood samples collected by this protocol is listed in Tables 7 and 8 under the column headed DLDW-VP for the D-Lead®-D-Wipe® Venous Stick Site Cleansing Protocol.

The venipuncture samples for both protocols were collected in lavender topped VACUTAINER® tubes containing EDTA as the anti-coagulant and 20 mL of blood was collected in each sample tube. All samples were shipped the same day via overnight service to the laboratory. They were analyzed at the same CLIA (Clinical Laboratory Improvement Amendments) licensed Laboratory on the same day, in the same run by GFAAS. One of the 30 subjects was not able to supply a second blood lead sample and is excluded from the data analysis. The analytical accuracy that can be achieved in the laboratory is +/−1 μg/dL.

### Results

The complete set of data is listed below in Table 7. All blood results are in micrograms of lead per deciliter of blood. The percent difference is calculated as:

$$\frac{[ DLDW \text{-} VP ] - [ CDC \text{-} VP ]}{[ DLDW \text{-} VP ]} * 100$$

TABLE 7
Venous Sample Test Data

| | Blood Lead μg/dL | | | |
|---|---|---|---|---|
| Test | DLDW- | CDC- | Difference | % |
| Subject #: | VP | VP | μg/dL | Difference |
| 1 | 24.5 | 27.8 | −3.3 | −13.5% |
| 3 | 32.0 | 34.0 | −2.0 | −6.3% |
| 4 | 18.1 | 19.2 | −1.1 | −6.1% |
| 5 | 18.4 | 19.2 | −0.8 | −4.3% |
| 6 | 18.9 | 21.4 | −2.5 | −13.2% |
| 7 | 15.7 | 22.7 | −7.0 | −44.6% |
| 8 | 24.6 | 25.5 | −0.9 | −3.7% |
| 9 | 40.4 | 43.8 | −3.4 | −8.4% |
| 10 | 16.2 | 18.6 | −2.4 | −14.8% |
| 11 | 24.7 | 26.5 | −1.8 | −7.3% |
| 12 | 26.7 | 28.4 | −1.7 | −6.4% |
| 13 | 15.8 | 18.7 | −2.9 | −18.4% |
| 14 | 21.7 | 23.5 | −1.8 | −8.3% |
| 15 | 32.8 | 36.3 | −3.5 | −10.7% |
| 16 | 24.9 | 28.6 | −3.7 | −14.9% |
| 17 | 14.9 | 16.2 | −1.3 | −8.7% |
| 18 | 23.1 | 24.7 | −1.6 | −6.9% |
| 19 | 19.4 | 26.0 | −6.6 | −34.0% |
| 20 | 15.6 | 19.9 | −4.3 | −27.6% |
| 21 | 18.9 | 21.0 | −2.1 | −11.1% |
| 22 | 29.1 | 32.0 | −2.9 | −10.0% |
| 23 | 25.9 | 27.8 | −1.9 | −7.3% |
| 24 | 19.3 | 22.6 | −3.3 | −17.1% |
| 25 | 24.6 | 25.5 | −0.9 | −3.7% |
| 26 | 32.2 | 32.0 | 0.2 | 0.6% |
| 27 | 11.6 | 11.6 | 0.0 | 0.0% |
| 28 | 36.9 | 36.1 | 0.8 | 2.2% |
| 29 | 27.9 | 28.0 | −0.1 | −0.4% |
| 30 | 9.4 | 15.1 | −5.7 | −60.6% |
| Averages | 22.9 | 25.3 | −2.4 | −12.6% |

In 26 of the 29 duplicate samples, the sample obtained utilizing the D-Lead®-D-Wipe® Stick Site Cleansing Protocol gave a lower blood lead value than the standard CDC Stick Site Cleansing Protocol. The reduction ranged from a reduction of 0.1 μg/dL (0.1%) to 5.7 μg/dL (61%) [at a blood lead of 9.4] and 6.6 μg/dL (34%) [at a blood lead of 19.4]. In one individual the result of both samples was identical, and for 2 individuals the CDC protocol gave a lower result, 0.2 μg/dL (−1%) and 0.8 μg/dL (−2%). However, these differences are entirely within the analytical accuracy of the laboratory method, +/−1.0 μg/dL, so the values are considered to be equal.

Of the 29 duplicate samples, 7 had results within the analytical accuracy of the analysis by GFAAS, +/−1 μg/dL. If we chart the remaining results for the 22 samples that differed by more than the difference of precision of the analysis, we see that the average blood lead level result is 16.2% less with the D-Lead®-D-Wipe® Stick Site Cleansing Protocol. All 22 of these samples were lower when the stick site was prepared with the D-Lead®-D-Wipe® Stick Site Cleansing Protocol. These 22 sample results are listed in Table 8.

TABLE 8
Blood Lead Results with Differences of more than the Level of Precision

| | Blood Lead μg/dL | | | |
|---|---|---|---|---|
| Test | DLDW- | CDC- | Difference | Test |
| Subject #: | VP | VP | μg/dL | Subject #: |
| 1 | 24.5 | 27.8 | −3.3 | −13.5% |
| 3 | 32.0 | 34.0 | −2.0 | −6.3% |
| 4 | 18.1 | 19.2 | −1.1 | −6.1% |
| 5 | 18.9 | 21.4 | −2.5 | −13.2% |
| 6 | 15.7 | 22.7 | −7.0 | −44.6% |
| 79 | 40.4 | 43.8 | −3.4 | −8.4% |
| 10 | 16.2 | 18.6 | −1.8 | −14.8% |
| 11 | 24.7 | 26.5 | −2.4 | −7.3% |
| 12 | 26.7 | 28.4 | −1.7 | −6.4% |
| 13 | 15.8 | 18.7 | −2.9 | −18.4% |
| 14 | 21.7 | 23.5 | −1.8 | −8.3% |
| 15 | 32.8 | 38.3 | −3.5 | −10.7% |
| 16 | 24.9 | 28.6 | −3.7 | −14.9% |
| 17 | 14.9 | 16.2 | −1.3 | −8.7% |
| 18 | 23.1 | 24.7 | −1.6 | −6.9% |
| 19 | 19.4 | 26.0 | −6.6 | −34.0% |
| 20 | 15.6 | 19.9 | −4.3 | −27.6% |
| 21 | 18.9 | 21.0 | −2.1 | −11.1% |
| 22 | 29.1 | 32.0 | −2.9 | −10.0% |
| 23 | 25.9 | 27.8 | −1.9 | −7.3% |
| 24 | 19.3 | 22.6 | −3.3 | −17.1% |
| 30 | 9.4 | 15.1 | −5.7 | −60.6% |
| Averages: | 22.2 | 25.2 | 3.0 | −16.2% |

For the 22 samples (81.5% of the individuals) with a difference greater than the analytical accuracy of the analytical method, all showed a reduction in the measured blood lead level by an average 3.0 μg/dL or 16.2%.

### Discussion

The reduction of the blood lead result cannot be explained by suppression or reduced availability of the lead in the blood sample during the analysis, as spiked blood lead tests clearly show this does not occur. The results can only be explained by a reduction in sample contamination of the venous blood sample. The D-Lead®-D-Wipe® Stick Site Cleansing Protocol was significantly better than the CDC protocol at eliminating blood sample contamination from lead on and in the surface of the skin. This reduction in sample contamination was at least 12.6% of the measured value.

### Conclusion

Rigorous blood sample stick site cleaning with highly efficient, contaminant specific skin cleaners provides a method that significantly reduces the amount lead from sources not in the circulating blood stream as compared to the standard stick site protocol recommended for collecting blood lead samples. The data indicates that the D-Lead®-D-Wipe® Stick Site Cleansing Protocol reduces contamination of the sample from the skin during the blood sampling step.

6. Analysis and Comparison of the D-Lead®-D-Wipe® Stick Site Cleansing Protocol for Capillary Blood Lead Samples vs. the Centers for Disease Control Stick Site Cleansing Protocol for Capillary Blood Lead Samples

### Objective

The level of accuracy achieved by GFAAS analysis of blood lead specimens collected on filter paper using the D-Lead®-D-Wipe® Stick Site Cleansing Protocol for Capillary Blood Lead Samples was compared with the level of accuracy achieved by whole blood capillary tube specimens analyzed by Inductively Coupled Mass Spectroscopy (ICMS). Then these results were compared with all other types of capillary blood lead specimen collection and analysis, using the current CDC—recommended stick site cleansing and preparation protocol.

### Background

It is generally accepted that the primary cause of falsely elevated capillary blood lead test results (whole blood or dry blood on filter paper) is pro-analytic contamination of the specimen by lead. In an effort to mitigate this threat to the accuracy and reliability of capillary blood lead testing, we investigated the use of the lead and metal removal skin cleaning products disclosed in this patent application in conjunction with capillary blood lead specimen collection.

In the effort to mitigate the blood sample contamination that has been prevalent (reported in the literature to be as high as 77%) in Capillary Blood Lead Screening Programs, we began working in conjunction with a CLIA licensed laboratory in mid 2004. At this time we began supplying and the laboratory began mandating the use of D-Lead® Deluxe Whole Body Wash and Shampoo and D-Wipe® Towels as part of the specimen collection protocol for collecting capillary blood samples collected with their Filter Paper Quantitative Blood Lead Test. This protocol is referred to as: The D-Lead®/D-Wipe® Capillary Stick Site Cleansing Protocol (DLDW-CP).

At approximately the same time that the mandated use of the D-Lead®/D-Wipe® Capillary Stick Site Cleansing Protocol was incorporated into the specimen collection protocol, this clinical laboratory was awarded a contract by a State Department of Health to analyze all public health blood lead specimens collected in the state. As a result of this contract, they performed GFAAS analysis of all blood lead specimens collected in Mississippi State's County Department of Health Clinics between Jul. 1, 2004 and Jul. 20, 2005. Specimens were collected by public health nurses on filter paper using the D-Lead®/D-Wipe® Capillary Stick Site Cleansing Protocol. This protocol involved thoroughly washing the stick site with D-Lead® Deluxe Whole Body Wash followed by a thorough rinse, then scrubbing the stick site with a D-Wipe® Towel, and wiping the stick site with an alcohol pad prior to making the stick.

Early in 2005, the state determined that its own Department of Public Health Laboratory would begin performing blood lead analysis for all specimens effective Jul. 1, 2005. For the period from Jul. 1, 2005 through Feb. 22, 2006 all public health specimens for the same state consisted of whole blood collected in capillary tubes. These specimens were analyzed by the state public health laboratory using Inductively Coupled Mass Spectroscopy. It is reasonable to assume that essentially the same group of public health nurses collected the specimens in 2005/2006 as collected the filter paper specimens in 2004/2005. It is presumed (but not known) that the standard CDC-recommended stick site cleansing and preparation protocol was used prior to the collection of the 2005/2006 whole blood capillary tube specimens. This protocol involves washing the stick site with soap and water and wiping the stick site with an alcohol pad prior to making the stick.

The Filter Paper blood lead specimens collected between Jul. 1, 2004 and Jul. 20, 2005 using the D-Lead®/D-Wipe® Capillary Stick Site Cleansing Protocol are compared with the whole blood capillary tube specimens collected using the CDC standard stick site cleansing and preparation protocol and analyzed by the state public health laboratory since Jul. 1, 2005 through Feb. 22, 2005.

### Sources of Data

Data for the Filter Paper sample results collected with the D-Lead®/D-Wipe® Capillary Stick Site Cleansing Protocol was drawn from the Laboratory's Medical Database. All of the DLDW-CP data cited is “as reported” to the State Department of Public Health. Data for all other laboratories and for the state public health laboratory was provided by the State Department of Public Health in response to a formal request for public documents. The data requested and provided was for all elevated capillary blood screening test results and the result of any subsequent follow up confirmation test.

### Discussion

In 2004/2005, a total of 14,413 specimens were submitted as capillary blood filter paper samples by the state's 88 testing sites to the contracted laboratory. The testing supplies were supplied by this same laboratory and included the D-Lead® Deluxe Skin Cleaner and D-Wipe® Towels. The written specimen collection procedure incorporated the D-Lead®/D-Wipe® Stick Site Cleansing Protocol. Of the total specimens submitted, 273 specimens (1.89%) were rejected by the laboratory. (Specimens are rejected when there is insufficient blood on the filter paper (‘QNS’-Quantity Not Sufficient), or the specimen does not meet sample quality requirements). The remaining 14,140 specimens were analyzed by GFAAS. This analysis yielded 362 results ≧10 μg/dL (2.56% of total specimens analyzed). Eighty four (84) of these elevated results were eliminated from the study because they were not confirmed by a subsequent venous test. An additional 74 elevated results were eliminated from the study because a confirmatory venous analysis was not performed within 90 days of capillary specimen analysis.

Of the remaining 204 elevated results, 94 (46.1%) met the defined accuracy criteria, and 110 (53.9%) did not meet the defined accuracy criteria.

Of the total of 13,982 blood lead test specimens in the study, 13,872 (99.21/%) met the defined accuracy criteria, 110 (0.79%) did not meet the defined accuracy criteria.

Two significant findings emerged from the data analysis.
1. Perfect accuracy (100%) was achieved by 41 of the 88 collection sites (46.6% of total). That is to say, 100% of the specimens they submitted met accuracy criteria. These sites submitted 3,763 specimens (26.18% of total).
2. All 110 samples that did not meet the accuracy criteria were submitted by 57 of the 88 sites. That is to say: Even the 57 sites that submitted one or more inaccurate samples had a high accuracy rating of 98.92%

Therefore, possible specimen contamination issues were confined to 53.94% of total sites and 73.82% of total specimens submitted. The largest site submitted 619 specimens of which 617 (99.68%) met accuracy criteria. The smallest site submitted 3 specimens, all of which met accuracy criteria.

Accuracy by site ranged from a high of 100% to a low of 95.24%. The ten largest sites submitted a total of 4,432 specimens, of which 4,391 (99.07%) met accuracy criteria. The ten smallest sites submitted a total of 147 specimens, of which 145 (98.64%) met accuracy criteria. Eight of the ten smallest sites had perfect accuracy records. The 15 sites (193%) with the lowest level of accuracy submitted 1,900 specimens of which 47 were inaccurate. These 15 sites submitted 42.7% of all of the inaccurate specimens

This suggests variability in the implementation and use of the mandated D-Lead® D-Wipe® Stick Site Cleansing Protocol by different sites.

### Results

Three sets of data have been compared:

1. Filter Paper specimens collected by the Mississippi state department of health in 2004/2005 who were supplied with the D-Lead®/D-Wipe® skin cleansing supplies and the D-Lead®/D-Wipe® Capillary Stick Site Cleansing and Prep Protocol and analyzed by GFAAS.

2. Whole blood capillary tube specimens collected in 2005/2006 by the same State Department of Public Health (and, presumably, the same collection staff) with other stick site cleansing and prep protocol and analyzed by the state's public health laboratory using Inductively Coupled Mass Spectroscopy (ICMS).

3. Specimens from the same state analyzed by all laboratories other than the laboratory listed in item #1 above in 2005/2006 using other stick site cleansing and prep protocol. This group includes all other laboratories who analyzed blood lead samples for public or private health care provider in the state during the year, by all methods, and this set of specimens is assumed to include alternative filter paper, whole blood capillary tube, and LeadCare®. The analysis of the blood lead samples in this set would include all recognized blood lead testing methods, which are analysis by GFAAS, ICMS, ASV and LeadCare® ASV.

1. Filter Paper Test with the D-Lead®/D-Wipe® Capillary Stick Site Cleansing Protocol

Time Period—Jul. 1, 2004 through Jul. 20, 2005-385 days

Specimen Collection Protocol—Dried blood on filter paper

Stick Site Cleansing and Prep Protocol—Wash with D-Lead® Deluxe, rinse, dry, wipe with D-Wipe® Towel, wipe with alcohol wipe, stick, collect

Analysis—GFAAS

TABLE 9
Summary of Testing with Filter Paper and D-Lead ®/D-
Wipe ® Capillary Stick Site Cleansing Protocol

| | |
|---|---|
| Total Filter Paper Specimens Submitted | 14,413 |
| Rejected Specimens (.0189) | 273 |
| Elevated Results with No Confirmatory Test | 84 |
| Elevated Results With ≧ 90-Day Confirmation. | 74 |
| Total Filter Paper DLDW-C P Results Studied | 13,982 |

TABLE 10
Accuracy of Filter Paper Tests using D-Lead ®/D-Wipe ®
Capillary Stick Site Cleansing Protocol

| | Number | % |
|---|---|---|
| Total Elevated FP Results With Confirmation ≦ 90 | 204 | 100% |
| Days | | |
| No. of Elevated Results With Confirmatory Venous | 94 | 46.1% |
| Results ≦ Days Meeting Accuracy Criteria | | |
| No. of Elevated Results With Confirmatory Venous | 110 | 53.9% |
| Results ≦ Days Not Meeting Accuracy Criteria | | |
| No. of Total Specimens Submitted Meeting Accuracy | 13,872 | 99.21% |
| Criteria | | |
| No. of Total Specimens Submitted Not Meeting | 110 | 0.79% |
| Accuracy Criteria | | |

2. Capillary Tube Test by State Public Health Laboratory Using CDC Capillary Specimen Collection Protocol
Time Period—Jul. 8, 2005 through Feb. 22, 2006-230 days
Specimen Collection—Whole Blood Capillary Tube
Stick Site Cleansing and Prep Protocol—CDC-recommended—wash soap/alcohol wipe/stick/collect
Methodology—ICMS

TABLE 11
Summary of Testing Capillary Tube Samples with Standard CDC
Capillary Stick Site Cleansing Protocol

| | | |
|---|---|---|
| No. of Elevated Capillary Specimens With | 44 | 100% |
| Confirmatory Venous Results ≦ 90 Days | | |
| No. of Elevated Capillary Specimens With | 17 | 36.8% |
| Confirmatory Venous Results ≦ Days Meeting | | |
| Accuracy Criteria | | |
| No. of Elevated Capillary Specimens With | 27 | 61.4% |
| Confirmatory Venous Results ≦ Days Not | | |
| Meeting Accuracy Criteria | | |

3. All Laboratories & Methods using Standard CDC capillary Stick Site Cleansing Protocol
Time Period—Jul. 1, 2005 through Mar. 7, 2006-250 days
Specimen Collection Protocol Alternative FP, Whole Blood Capillary Tube, LeadCare®
Stick Site Cleansing and Prep Protocol—CDC recommended—wash soap/alcohol wipe/stick/collect
Methodology—Undetermined combination of ICMS, GFAAS, ASV, LeadCare® ASV

TABLE 12
Summary of All Lab Testing Capillary Samples with Standard CDC
Capillary Stick Site Cleansing Protocol

| | | |
|---|---|---|
| No. of Elevated Capillary Specimens With | 172 | 100% |
| Confirmatory Venous Results ≦ 90 Days | | |
| No. of Elevated Capillary Specimens With | 71 | 41.3% |
| Confirmatory Venous Results ≦ 90 Days Meeting | | |
| Accuracy Criteria | | |
| No. of Elevated Capillary Specimens With | 101 | 58.7% |
| Confirmatory Venous Results ≦ 90 Days Not | | |
| Meeting Accuracy Criteria | | |

TABLE 13
Comparison of elevated Capillary Results Meeting Accuracy Criteria

| | | | All |
|---|---|---|---|
| | Filter Paper | State Public Health | Laboratories & |
| | with D-Lead ® / | Laboratory with whole | Methods using |
| | D-Wipe ® | blood capillary tube | Standard CDC |
| | Capillary Stick | and Standard CDC | Capillary Stick |
| | Site Cleansing | Capillary Stick Site | Site Cleansing |
| Methodology | Protocol | Cleansing Protocol | Protocol |
| % Accurate | 46.1 % | 38.64 % | 41.3% |
| Results | | | |

The Filter Paper & D-Lead®/D-Wipe® Protocol had 19.30% greater accuracy than the State Laboratory with the CDC Protocol.

The Filter Paper & D-Lead®/D-Wipe® Protocol had 11.63% greater accuracy than all other Laboratories and Methods with the CDC Protocol.

### Conclusion

Given the definition of capillary blood lead testing accuracy, and the large volume of data, including data from comparative filter paper and capillary tube specimens collected by the same individuals:

1. Even though the Filter Paper method introduces an additional drying and handling step with the inherent opportunity for additional sample contamination, that, in actual practice, the use of the D-Lead®/D-Wipe® Capillary Stick Site Cleansing Protocol achieved a higher level of accuracy than was obtained with the collection of whole blood capillary tube specimens using other standard stick site cleansing and prep protocols and ICMS analysis.

2. That, in actual practice, the use of the D-Lead®/D-Wipe® Capillary Stick Site Cleansing Protocol with analysis by GFAAS provides a higher level of accuracy than can be achieved on a statewide basis, by all alternative forms of capillary blood lead specimen collection and analysis methods using other standard stick site cleansing and prep protocols.

In addition to the methods discussed previously, the following are additional preferred methods of the present invention.

For capillary blood samples collected from the finger: If water is available: Wet hands, apply skin cleanser of the liquid type described as Type A, wash, rinse with clean water, then scrub the stick site with the specially formulated wipe described herein, then the alcohol wipe.

For capillary blood samples collected from the finger: If water is not available: Apply a liquid skin cleanser of the type described as Type B, wash, wipe cleanser off with a cotton or paper towel, then scrub the stick site with the specially formulated wipe described herein, then the alcohol wipe.

For capillary blood samples collected from the ear lobe, toe or heel, after washing the hands as described above, wash the stick site with the appropriate skin cleanser, then wipe with the premoistened towel, then the alcohol wipe.

For venous blood samples collected from the forearm: If water is available: Wet hands and arms, apply skin cleanser of the liquid type described as Type A, wash hands and arms to a point above the stick site, rinse with clean water, then repeat by washing the blood sample stick site and rinsing with clean water. Then scrub the stick site with the specially formulated wipe described here, then the alcohol wipe.

For venous blood samples collected from the forearm: If water is not available: Apply a liquid skin cleanser of the type described as Type B, wash hands and arms to a point above the stick site, wipe cleanser off with a dry cotton or paper towel, then wash the stick site with the Type B skin cleaner and wipe cleaner off with a dry cotton or paper towel, and then scrub the stick site with the specially formulated wipe described here, then the alcohol wipe.

The skin cleansers and/or premoistened wipe can also incorporate a skin disinfectant to eliminate the alcohol wipe step.

Various alternatives are contemplated as being within the scope of the following claims particularly pointing out and distinctly claiming the subject matter regarded as the invention.

I claim:

1. A method for performing a blood contaminant test, the method comprising the steps of:
a) cleaning a blood sampling site on an individual prior to collection of a blood sample by:
i) applying a metal contaminant-removing cleanser to a blood sampling site to remove the metal contaminant to be measured in the blood from the surface of the skin, and from subsurface areas of the site, including the pores, sweat ducts, hair follicles and sebaceous glands at the site, the cleanser comprising at least one surfactant, an anti-static component and at least one metal contaminant-removing agent present in an amount of between 0.1% w/w to about 25% w/w of the cleanser;
ii) dissipating a static charge on the metal contaminant holding the metal contaminant on the skin surface and subsurface areas; and
iii) removing the skin cleanser along with the metal contaminant from the site while drying the site using a fabric substrate, wherein the fabric substrate in conjunction with the cleanser binds the contaminant to the fabric;
b) obtaining a venous blood sample at the cleaned sampling site; and
c) performing an analysis on the blood sample obtained from the site to determine the amount of the contaminant in the blood sample, wherein the accuracy of the sampling and analysis of the blood sample for elevated levels of the contaminant in the blood sample is +/−3ug/100 mL.

2. The method of claim 1 wherein the at least one contaminant-removing agent is a chelating agent.

3. The method of claim 1 wherein the at least one contaminant-removing agent is a phosphonate.

4. The method of claim 1 wherein the at least one contaminant-removing agent is a combination of a chelating agent and a phosphonate.

5. The method of claim 1 wherein the at least one surfactant is selected from the group consisting of at least one cationic surfactant, at least one anionic surfactant, at least one amphoteric surfactant and combinations thereof.

6. The method of claim 1 wherein the at least one contaminant-removing agent is a terpene.

7. The method of claim 1 wherein the at least one contaminant-removing agent is a combination of a terpene and a phosphonate.

8. The method of claim 1 wherein the cleanser comprises two or more of:
a) a terpene;
b) a surfactant;
c) an alkanolamine;
d) an amine oxide; and
e) a phosphonate.

9. The method of claim 1 further comprising the step of scrubbing the site with a contaminant-removing substrate moistened with a contaminant-removing solution after removing the cleanser.

10. The method of claim 9 wherein the step of scrubbing the site with the substrate comprises simultaneously exfoliating dead cells on the site.

11. The method of claim 1 wherein the cleanser further comprises an abrasive and further comprising the step of exfoliating dead cells from the site simultaneously with applying the cleanser to the site.

12. The method of claim 1 wherein the step of applying the skin cleanser comprises:
a) applying water to the site; and
b) applying the skin cleanser to the site.

13. The method of claim 12 further comprising the steps of:
a) rinsing the site with water after applying the cleanser to the site; and
b) scrubbing the site with a contaminant-removing substrate moistened with a contaminant-removing solution after rinsing the site.

14. The method of claim 13 further comprising the steps of:
a) reapplying the cleanser to the site after rinsing the site and before scrubbing the site; and
b) re-rinsing the site with water prior to scrubbing the site.

15. The method of claim 1 wherein the step of applying the cleanser to the site does not include water.

16. The method of claim 15 wherein the step of removing the cleanser from the site does not include water.

17. The method of claim 1 further comprising the step of disinfecting the site after removing the cleanser from the site.

18. The method of claim 1 wherein the contaminant to be removed is an inorganic metal.

19. The method of claim 18 wherein the inorganic metals is selected from the group consisting of calcium, magnesium, lead, mercury, cadmium, manganese, strontium, barium, iron, cobalt, nickel, copper, zinc, thorium, radium and uranium.

20. A method for improving the blood flow at a blood sampling site, the method comprising the steps of:
a) applying a contaminant-removing cleanser to the site to remove the contaminant to be measured in the blood from the surface of the skin, the pores, sweat ducts, hair follicles and sebaceous glands at the site, the cleanser comprising at least one surfactant, an anti-static component, at least one metal contaminant-removing agent present in an amount of between 0.1% w/w to about 25% w/w of the cleanser and at least one calcium-removing agent present in an amount of between 0.1% w/w to about 25% w/w of the cleanser, and
b) removing the skin cleanser from the site.

21. The method of claim 20 wherein the method does not include water.

22. A method for performing a blood contaminant test, the method comprising the steps of:
a) cleaning a blood sampling site on an individual prior to collection of a blood sample by:
i) applying a metal contaminant-removing cleanser to a blood sampling site to remove the metal contaminant to be measured in the blood from the surface of the skin, and from subsurface areas of the site, including the pores, sweat ducts, hair follicles and sebaceous glands at the site, the cleanser comprising at least one surfactant, an anti-static component and at least one metal contaminant-removing agent present in an amount of between 0.1% w/w to about 25% w/w of the cleanser,
ii) dissipating a static charge on the metal contaminant holding the metal contaminant on the skin surface and subsurface areas; and
iii) removing the skin cleanser along with the metal contaminant from the site while drying the site using a fabric substrate, wherein the fabric substrate in conjunction with the cleanser binds the contaminant to the fabric;
b) obtaining a venous blood sample at the cleaned sampling site; and
c) performing an analysis on the blood sample obtained from the site to determine the amount of the contaminant in the blood sample, wherein the accuracy of the sampling and analysis of the blood sample for elevated levels of the contaminant in the blood sample is +/−7.5%.

23. The method of claim 22 wherein the at least one surfactant, an anti-static component and at least one metal contaminant-removing agent are different from one another.

24. The method of claim 1 wherein the at least one surfactant, an anti-static component and at least one metal contaminant-removing agent are different from one another.
